(19) United States
(12) Patent Application Publication
(10) Pub. No.: US 2008/0032924 A1
(43) Pub. Date: Feb. 7, 2008

(54) Antifungal Peptides

(76) Inventors: Peter David East, Australian Capital Territory (AU); Susan Elizabeth Brown, Australian Capital Territory (AU)

Correspondence Address: COOPER & DUNHAM, LLP, 1185 AVENUE OF THE AMERICAS, NEW YORK, NY, 10036, US

(21) Appl. No.: 10/590,539

(22) PCT Filed: Feb. 23, 2005
(86) PCT No.: PCT/AU05/00234

§ 371 Date: May 30, 2007

(30) Foreign Application Priority Data
Feb. 24, 2004 (AU) 2004900938

(51) Int. Cl. A61K 38/00; A01H 5/00; A01K 67/00; C07K 14/00; C12N 15/00; C12N 5/04; C12P 21/04; C12N 5/06; C12N 15/11; C07K 16/18; A61P 43/00; A61K 31/70

(52) U.S. Cl. 514/12; 435/320.1; 435/325; 435/410; 435/69.1; 514/44; 530/324; 530/387.9; 536/23.1; 800/13; 800/298

(57) ABSTRACT

The present invention provides antifungal and antibacterial peptides, especially antifungal peptides obtained from insect species, particularly lepidopterans. The present invention also provides methods of using these antifungal peptides to treat or prevent fungal growth for a variety of purposes such as; protecting plants from fungal infections, treating fungal infections of animals, especially humans, and prevention of food spoilage. In particular, the invention provides transgenic plants that produce an antifungal peptide, said plants having increased resistance to fungal infections/growth.

### FIELD OF THE INVENTION

The present invention relates to antifungal peptides, especially antifungal peptides obtained from insect species, particularly lepidopterans. The present invention also provides methods of using these antifungal peptides to treat or prevent fungal growth for a variety of purposes such as; protecting plants from fungal infections, treating fungal infections of animals, especially humans, and prevention of food spoilage.

### BACKGROUND OF THE INVENTION

Fungi are eukaryotic cells that may reproduce sexually or asexually and may be biphasic, with one form in nature and a different form in the infected host. Fungal infections of plants and animals are a significant problem in the fields of agriculture, medicine and food production/storage. Fungal infections are becoming a major concern for a number of reasons, including the limited number of antifungal agents available, the increasing incidence of species resistant to older antifungal agents, and the growing population of immunocompromised patients at risk for opportunistic fungal infections.

Fungal diseases of humans are referred to as mycoses. Some mycoses are endemic, where infection is acquired in the geographic area that is the natural habitat of that fungus. These endemic mycoses are usually self-limited and minimally symptomatic. Some mycoses are chiefly opportunistic, occurring in immunocompromised patients such as organ transplant patients, cancer patients undergoing chemotherapy, burn patients, AIDS patients, or patients with diabetic ketoacidosis.

Fungi cause many diseases of plants such as, but not limited to, mildews, rots, rusts, smuts, and wilts etc. For example, soilborne fungal phytopathogens cause enormous economic losses in the agricultural and horticultural industries. In particular, Rhizoctonia solani is one of the major fungal phytopathogens exhibiting strong pathogenicity; it is associated with seedling diseases as well as foliar diseases such as seed rot, root rot, damping-off, leaf and stem rot of many plant species and varieties, resulting in enormous economic losses. Another example is Phytophthora capsici which is a widespread and highly destructive soilborne fungal phytopathogen that causes root and crown rot as well as the aerial blight of leaves, fruit, and the stems of peppers (Capsicum annuum L.).

Plant fungus infection is a particular problem in damp climates and may become a major concern during crop storage. Plants have developed a certain degree of natural resistance to pathogenic fungi; however, modem growing methods, harvesting and storage systems frequently provide a favorable environment for plant pathogens.

Antifungal agents include polyene derivatives, such as amphotericin B and the structurally related compounds nystatin and pimaricin. Furthermore, antifungal peptides have been isolated from a variety of naturally occurring sources (DeLucca and Walsh, 1999). However, there is a need for the identification of further compounds with antifungal activity for use in medical, agricultural and industrial related applications to control and/or prevent fungal growth.

### SUMMARY OF THE INVENTION

The present inventors have isolated and characterized new antimicrobial, particularly antifungal, peptides. Accordingly, in a first aspect the present invention provides a substantially purified peptide which comprises a sequence selected from the group consisting of:
i) an amino acid sequence as provided in SEQ ID NO:4,
ii) an amino acid sequence which is at least 60% identical to SEQ ID NO:4,
iii) an amino acid sequence as provided in SEQ ID NO:5,
iv) an amino acid sequence which is at least 80% identical to SEQ ID NO:5,
v) an amino acid sequence as provided in SEQ ID NO:48,
vi) an amino acid sequence which is at least 70% identical to SEQ ID NO:48,
vii) an amino acid sequence as provided in SEQ ID NO:53,
viii) an amino acid sequence which is at least 70% identical to SEQ ID NO:53,
ix) a biologically active fragment of any one of i) to viii), and
x) a precursor comprising the amino acid sequence according to any one of i) to ix),
   wherein the peptide, or fragment thereof, exhibits antifungal and/or antibacterial activity.

In a preferred embodiment of the first aspect, the peptide is, where relevant, at least 65%, more preferably at least 70%, more preferably at least 75%, more preferably at least 80%, more preferably at least 85%, more preferably at least 90%, more preferably at least 92%, more preferably at least 95%, more preferably at least 97%, and even more preferably at least 99% identical to the sequence provided in SEQ ID NO:4, SEQ ID NO:5, SEQ ID NO:48 or SEQ ID NO:53.

Preferably, the precursor of SEQ ID NO:4 is SEQ ID NO:1 or SEQ ID NO:2, the precursor of SEQ ID NO:5 is SEQ ID NO:3, the precursor of SEQ ID NO:48 is SEQ ID NO:47, and the precursor of SEQ ID NO:53 is SEQ ID NO:52.

Preferably, the peptide can be purified from an insect. More preferably, the peptide can be purified from a lepidopteran insect. More preferably, the peptide can be purified from a lepidopteran insect of the family Pyralidae. More preferably, the peptide can be purified from a Galleria sp. Even more preferably, the peptide can be purified from Galleria mellonella.

In a particularly preferred embodiment, the peptide can be purified from an insect which has been exposed to a fungal or bacterial infection. In the case of lepidpoterans, it is preferred that the peptide can be purified from last instar larvae that have been exposed to bacteria such as, but not limited to, Escherichia coli and/or Micrococcus luteus.

In another embodiment, it is preferred that the peptide has a molecular weight of between about 4.5 kDa to about 3.3 kDa. More preferably, the peptide has a molecular weight of about 4.3 kDa, about 4.0 kDa, or about 3.6 kDa.

In yet a further preferred embodiment, the peptide comprises an N-terminal amphipathic (at least relative to the C-terminus) region which includes a helical structure, a C-terminal hydrophobic (at least relative to the N-terminus) region which also includes a helical structure and an acidic residue, and a charged C-terminal tail.

In a further preferred embodiment, the peptide comprises the amino acid sequence;

(SEQ ID NO:62)

Xaa₁ Lys Xaa₂ Xaa₃ Xaa₄ Xaa₅ Ala Ile Lys Lys Gly
Gly Xaa₆ Xaa₇ Ile Xaa₈ Lys Xaa₉ Xaa₁₀ Xaa₁₁ Xaa₁₂
Xaa₁₃ Xaa₁₄ Xaa₁₅ Ala Xaa₁₆ Thr Ala His Xaa₁₇
Xaa₁₈ Xaa₁₉ Xaa₂₀ Xaa₂₁ Xaa₂₂ Xaa₂₃ Xaa₂₄ Xaa₂₅
Xaa₂₆ Xaa₂₇ Xaa₂₈ Xaa₂₉ Xaa₃₀.

Preferably, Xaa₁ is Gly, Pro, Ala or absent, more preferably Gly or absent.

Preferably, Xaa₂ is Ile, Val, Ala, Leu, Met or Phe, more preferably Ile or Val.

Preferably, Xaa₃ is Pro, Gly, Asn, Gln or His, more preferably Pro or Asn.

Preferably, Xaa₄ is Ile, Val, Ala, Leu, Met or Phe, more preferably Ile or Val.

Preferably, Xaa₅ is Lys, Arg, Gly, Pro, Ala, Asn, Gln or His, more preferably Lys, Gly or Asn.

Preferably, Xaa₆ is Gln, Asn, His, Lys or Arg, more preferably Gln or Lys.

Preferably, Xaa₇ is Ile, Val, Ala, Leu or Gly, more preferably Ile or Ala.

Preferably, Xaa₈ is Gly, Pro, Ala, Lys or Arg, more preferably Gly or Lys.

Preferably, Xaa₉ is Val, Leu, Ile, Gly, Pro or Ala, more preferably Ala or Gly.

Preferably, Xaa₁₀ is Ile, Val, Met, Ala, Phe or Leu, more preferably Leu or Phe.

Preferably, Xaa₁₁ is Arg, Lys, Gly, Pro or Ala, more preferably Arg, Gly or Lys.

Preferably, Xaa₁₂ is Gly, Pro, Ala, Val, Ile, Leu, Met, or Phe, more preferably Gly or Val.

Preferably, Xaa₁₃ is Ile, Leu, Val, Ala, Met or Phe, more preferably Val, Ile or Leu.

Preferably, Xaa₁₄ is Asn, Gln, His, Gly, Pro, Ala, Ser or Thr, more preferably Asn, Gly or Ser.

Preferably, Xaa₁₅ is Ile, Val, Ala, Leu or Gly, more preferably Ile or Ala.

Preferably, Xaa₁₆ is Ser, Thr, Gly, Pro or Ala, more preferably Ser or Gly.

Preferably, Xaa₁₇ is Asp or Glu.

Preferably, Xaa₁₈ is Ile, Leu, Val, Ala, Met or Phe, more preferably Ile or Val.

Preferably, Xaa₁₉ is Ile, Leu, Val, Ala, Tyr, Trp or Phe, more preferably Ile or Tyr.

Preferably, Xaa₂₀ is Ser, Thr, Asn, Gin, His, Glu or Asp, more preferably Ser, Asn or Glu.

Preferably, Xaa₂₁ is Gln, Asn or His, more preferably Gln or His.

Preferably, Xaa₂₂ is Phe, Leu, Val, Ala, Ile or Met, more preferably Phe, Val or Ile.

Preferably, Xaa₂₃ is Lys or Arg.

Preferably, Xaa₂₄ is Pro, Gly, Asn, Gln or His, more preferably Pro or Asn.

Preferably, Xaa₂₅ is Lys or Arg.

Preferably, Xaa₂₆ is Lys, Arg, His, Asn or Gln, more preferably Lys, His, Gln or Arg.

Preferably, Xaa₂₇ is Lys, Arg, His, Asn, Gln or absent, more preferably Lys, His or absent.

Preferably, Xaa₂₈ is Lys, Arg or absent, more preferably Lys or absent.

Preferably, Xaa₂₉ is Asn, Gln, His or absent, more preferably Asn or absent.

Preferably, Xaa₃₀ is His, Asn, Gln or absent, more preferably His or absent.

In a further preferred embodiment, the lysine at position 17 of SEQ ID NO:62 can be substituted with a threonine reside.

Preferably, the peptide (or fragment thereof) exhibits antifungal activity. More preferably, the peptide exhibits antifungal activity against the Family of fungi selected from, but not limited to, the group consisting of: Nectriaceae, Pleosporaceae, Mycosphaerellaceae, Phyllachoraceae, Leptosphaeria, and Trichocomaceae. More preferably, the peptide exhibits antifungal activity against the Genera of fungi selected from, but not limited to, the group consisting of: Fusarium (also known in the art as Gibberella), Alternaria, Ascochyta, Colletotrichum, Leptosphaeria and Aspergillus. In a particularly preferred embodiment, the peptide exhibits antifungal activity against the Genera of fungi which infect plants selected from, but not limited to, the group consisting of: Alternaria; Ascochyta; Botrytis; Cercospora; Colletotrichum; Diplodia; Erysiphe; Fusarium; Gaeumanomyces; Helminthosporium; Leptosphaeria, Macrophomina; Nectria; Peronospora; Phoma; Phymatotrichum; Phytophthora; Plasmopara; Podosphaera; Puccinia; Puthium; Pyrenophora; Pyricularia; Pythium; Rhizoctonia; Scerotium; Sclerotinia; Septoria; Thielaviopsis; Uncinula; Venturia; and Verticillium. In a further preferred embodiment, the peptide exhibits antifungal activity against the fungi selected from the group consisting of: Fusarium graminearum, Fusarium oxysporum, Ascochyta rabiei, Candida albicans, C. parapsilosis, C. glabrata, C. krusei, C. tropicalis, Cryptococcus neoformans and Leptosphaeria maculans.

In a further aspect, the present invention provides a peptide according to the invention which is fused to at least one other polypeptide/peptide sequence.

In a preferred embodiment, the at least one other polypeptide/peptide is selected from the group consisting of: a polypeptide/peptide that enhances the stability of a peptide of the present invention, a polypeptide/peptide that assists in the purification of the fusion protein, a polypeptide/peptide which assists in the peptide of the invention being secreted from a cell (particularly a plant cell), and a polypeptide/peptide which renders the fusion protein non-toxic to a fungus or a bacteria but which can be processed, for example by proteolytic cleavage, to produce an antifungal peptide of the invention.

In another aspect, the present invention provides an isolated polynucleotide, the polynucleotide comprising a sequence selected from the group consisting of:
i) a sequence of nucleotides provided in SEQ ID NO:9 or SEQ ID NO:10;
ii) a sequence of nucleotides provided in SEQ ID NO:11;
iii) a sequence of nucleotides provided in SEQ ID NO:12;
iv) a sequence of nucleotides provided in SEQ ID NO:13;
v) a sequence of nucleotides provided in SEQ ID NO:50;
vi) a sequence of nucleotides provided in SEQ ID NO:51;
vii) a sequence of nucleotides provided in SEQ ID NO:55;
viii) a sequence of nucleotides provided in SEQ ID NO:56;
ix) a sequence encoding a peptide of the invention;
x) a sequence of nucleotides which is at least 66% identical to SEQ ID NO:9, SEQ ID NO:10, or SEQ ID NO:12;
xi) a sequence of nucleotides which is at least 71% identical to SEQ ID NO:11 or SEQ ID NO:13;
xii) a sequence of nucleotides which is at least 62% identical to SEQ ID NO:50, or SEQ ID NO:51;
xiii) a sequence of nucleotides which is at least 62% identical to SEQ ID NO:55, or SEQ ID NO:56; and
xiv) a sequence which hybridizes to any one of (i) to (viii) under high stringency conditions.

Preferably, the polynucleotide encodes a peptide with antifungal and/or antibacterial activity.

In a preferred embodiment, the polynucleotide is, if relevant, at least 65%, more preferably at least 70%, more preferably at least 75%, more preferably at least 80%, more preferably at least 85%, more preferably at least 90%, more preferably at least 92%, more preferably at least 95%, more preferably at least 97%, and even more preferably at least 99% identical to SEQ ID NO:9, SEQ ID NO:10, SEQ ID NO:11, SEQ ID NO:12, SEQ ID NO:13, SEQ ID NO:50, SEQ ID NO:51, SEQ ID NO:55 or SEQ ID NO:56.

Preferably, the polynucleotide can be isolated from an insect. More preferably, the polynucleotide can be isolated from a lepidopteran insect. More preferably, the polynucleotide can be isolated from lepidopteran insect of the family Pyralidae. More preferably, the polynucleotide can be isolated from a Galleria sp. Even more preferably, the polynucleotide can be isolated from Galleria mellonella.

In another embodiment, the polynucleotide comprises a sequence provided as SEQ ID NO:6, SEQ ID NO:7, SEQ ID NO:8, SEQ ID NO:49 or SEQ ID NO:54.

Furthermore, the present invention provides a suitable vector for the replication and/or expression of a polynucleotide according to the invention. Thus, also provided is a vector comprising a polynucleotide of the invention.

The vectors may be, for example, a plasmid, virus, transposon or phage vector provided with an origin of replication, and preferably a promotor for the expression of the polynucleotide and optionally a regulator of the promotor. The vector may contain one or more selectable markers, for example an ampicillin resistance gene in the case of a bacterial plasmid or a neomycin resistance gene for a mammalian expression vector. The vector may be used in vitro, for example for the production of RNA or used to transfect or transform a host cell.

In another aspect the present invention provides a host cell comprising a vector, or polynucleotide, of the invention.

Preferably, the host cell is an animal, yeast, bacterial or plant cell. More preferably, host cell is a plant cell.

In a further aspect, the present invention provides a process for preparing a peptide according to the first aspect, the process comprising cultivating a host cell according to the invention under conditions which allow expression of the polynucleotide encoding the peptide, and recovering the expressed peptide.

The present invention also provides peptides produced by a process of the invention.

In a further aspect, the present invention provides a composition comprising a peptide, a polynucleotide, a vector, an antibody or a host cell of the invention, and one or more acceptable carriers.

In an embodiment, the carrier is a pharmaceutically, veterinary or agriculturally acceptable carrier.

In yet another aspect, the present invention provides a method for killing, or inhibiting the growth and/or reproduction of a fungus, the method comprising exposing the fungus to a peptide of the invention.

As the skilled addressee would be aware, the fungus can be exposed to the peptide by any means known in the art. In one embodiment, the fungus is exposed to a composition comprising the peptide. In another embodiment, the fungus is exposed to a host cell producing the peptide.

Plants and non-human animals resistant to fungal infections can be produced by introducing a polynucleotide of the invention into the plant or animal such that the peptide is produced in the transgenic organism.

Accordingly, in another aspect, the present invention provides a transgenic plant, the plant having been transformed with a polynucleotide according to the present invention, wherein the plant produces a peptide of the invention.

The transgenic plant can be any species of plant, however, it is preferred that the plant is a crop plant. Examples of such crop plants include, but are not limited to, wheat, barley, rice, chickpeas, field peas and the like.

In a further aspect, the present invention provides a method of controlling fungal infections of a crop, the method comprising cultivating a crop of transgenic plants of the invention.

In addition, in another aspect, the present invention provides a transgenic non-human animal, the animal having been transformed with a polynucleotide according to the present invention, wherein the animal produces a peptide of the invention.

In a further aspect, the present invention provides a method of treating or preventing a fungal infection in a patient, the method comprising administering to the patient a peptide of the invention.

In addition, the present invention provides for the use of a peptide of the invention for the manufacture of a medicament for treating or preventing a fungal infection in a patient.

Also provided is an antibody which specifically binds a peptide of the first aspect. Such antibodies will be useful as markers for peptide production from transgenic systems such as transgenic plants. In addition, such antibodies may be useful in methods of purifying peptides of the invention from insect lysates and/or recombinant expression systems.

It is envisaged by the present inventors that the peptides of the invention also exhibit antibacterial activity. Thus, the present invention also provides a method for killing, or inhibiting the growth and/or reproduction of a bacteria, the method comprising exposing the bacteria to a peptide of the invention.

The bacteria can be gram-positive or gram-negative.

As the skilled addressee would be aware, the bacteria can be exposed to the peptide by any means known in the art. In one embodiment, the bacteria is exposed to a composition comprising the peptide. In another embodiment, the bacteria is exposed to a host cell producing the peptide.

In a further aspect, the present invention provides a method of controlling bacterial infections of a crop, the method comprising cultivating a crop of transgenic plants of the invention.

In a further aspect, the present invention provides a method of treating or preventing a bacterial infection in a patient, the method comprising administering to the patient a peptide of the invention.

In addition, the present invention provides for the use of a peptide of the invention for the manufacture of a medicament for treating or preventing a bacterial infection in a patient.

In a further aspect, the present invention provides a method for killing, or inhibiting the growth and/or reproduction of a fungus, the method comprising exposing the fungus to a peptide which comprises a sequence selected from the group consisting of:
i) an amino acid sequence comprising residues 25 to 67 of SEQ ID NO:14,
ii) an amino acid sequence as provided in SEQ ID NO:17,
iii) an amino acid sequence comprising residues 26 to 67 of SEQ ID NO:15,
iv) an amino acid sequence which is at least 75% identical to any one of i) to iii),
v) an amino acid sequence comprising residues 26 to 66 of SEQ ID NO:18,
vi) an amino acid sequence which is at least 50% identical to v), and
vii) a biologically active fragment of any one of i) to vi).

In a preferred embodiment, the peptide is, where relevant, at least 60%, more preferably at least 65%, more preferably at least 70%, more preferably at least 75%, more preferably at least 80%, more preferably at least 85%, more preferably at least 90%, more preferably at least 92%, more preferably at least 95%, more preferably at least 97%, and even more preferably at least 99% identical to the sequence of any one of i) to iii) or v).

Construction of a phylogenetic tree based on the ClustalW alignment of the mature peptide sequences (FIG. 9) indicated that GmmoricinA, GmmoricinC1, GmmoricinC2 and BmmoricinX were closely related and could be considered to cluster together as a sub-family of the moricins. Antifungal testing of two members of this sub-family (synthetic Gm-moricinA and Gm-moricinC2) suggested that this group of peptides had better antifungal activity than the synthetic B. mori moricin. Thus, in a particularly preferred embodiment, the peptide comprises a sequence selected from the group consisting of:
i) an amino acid sequence comprising residues 26 to 66 of SEQ ID NO:18,
ii) an amino acid sequence which is at least 50% identical to i), and
iii) a biologically active fragment of i) or ii).

Preferably, the peptide can be purified from an insect. More preferably, the peptide can be purified from a Lepidoptera. More preferably, the peptide can be purified from a Lepidoptera of a Family selected from the group consisting of Pyralidae, Noctuidae, Bombycidae, and Sphingidae.

In one embodiment, the peptide is provided as a precursor such as SEQ ID NO:14, SEQ ID NO:15, or SEQ ID NO:18 which is processed to produce the biologically active peptide.

As the skilled addressee would be aware, the fungus can be exposed to the peptide by any means known in the art. In one embodiment, the fungus is exposed to a composition comprising the peptide. In another embodiment, the fungus is exposed to a host cell producing the peptide. In yet another embodiment, the fungus is exposed to a transgenic plant producing the peptide.

In a further aspect, the present invention provides a method of controlling fungal infections of a crop, the method comprising cultivating a crop of transgenic plants which produce a peptide which comprises a sequence selected from the group consisting of:
i) an amino acid sequence comprising residues 25 to 67 of SEQ ID NO:14,
ii) an amino acid sequence comprising residues 25 to 66 of SEQ ID NO:16,
iii) an amino acid sequence as provided in SEQ ID NO:17,
iv) an amino acid sequence comprising residues 26 to 67 of SEQ ID NO:15,
v) an amino acid sequence which is at least 75% identical to any one of i) to iv),
vi) an amino acid sequence comprising residues 26 to 66 of SEQ ID NO:18,
vii) an amino acid sequence which is at least 50% identical to vi), and
viii) a biologically active fragment of any one of i) to vii).

Preferably, the peptide comprises a sequence selected from the group consisting of:
i) an amino acid sequence comprising residues 26 to 66 of SEQ ID NO:18,
ii) an amino acid sequence which is at least 50% identical to i), and
iii) a biologically active fragment of i) or ii).

In a preferred embodiment of the above aspect, the peptide is not SEQ ID NO:59, SEQ ID NO:60, SEQ ID NO:61, or fragment thereof with antifungal activity. In a further preferred embodiment, the peptide does not have an alanine residue at position 32 (position relative to that as shown in SEQ ID NO:62).

In a further aspect, the present invention provides a method of treating or preventing a fungal infection in a patient, the method comprising administering to the patient a peptide which comprises a sequence selected from the group consisting of:
i) an amino acid sequence comprising residues 25 to 67 of SEQ ID NO:14,
ii) an amino acid sequence as provided in SEQ ID NO:17,
iii) an amino acid sequence comprising residues 26 to 67 of SEQ ID NO:15,
iv) an amino acid sequence which is at least 75% identical to any one of i) to iii),
v) an amino acid sequence comprising residues 26 to 66 of SEQ ID NO:18,
vi) an amino acid sequence which is at least 50% identical to v), and
vii) a biologically active fragment of any one of i) to vi).

In addition, the present invention provides for the use of a peptide which comprises a sequence selected from the group consisting of:

i) an amino acid sequence comprising residues 25 to 67 of SEQ ID NO:14,
ii) an amino acid sequence as provided in SEQ ID NO:17,
iii) an amino acid sequence comprising residues 26 to 67 of SEQ ID NO:15,
iv) an amino acid sequence which is at least 75% identical to any one of i) to iii),
v) an amino acid sequence comprising residues 26 to 66 of SEQ ID NO:18,
vi) an amino acid sequence which is at least 50% identical to v), and
vii) a biologically active fragment of any one of i) to vi)
   for the manufacture of a medicament for treating or preventing a fungal infection in a patient.

Also provided is a kit comprising a peptide, polynucleotide, vector, host cell, antibody or composition of the present invention.

In a further embodiment, the kit comprises other antimicrobial compounds such as those provided as SEQ ID NO's 14 to 18, or 57 to 61, or biologically active fragments thereof.

Preferably, the kit further comprises information and/or instructions for use of the kit.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1. Sequence alignment of the nucleotide sequences of two Gm-moricinA cDNA clones (GmmoriAe—SEQ ID NO:6; GmmoriAc—SEQ ID NO:7). The sequence of GmmoriAe was the same as GmmoriAa and GmmoriAd, however, the latter two were shorter at the 5′ end. Non conserved nucleotides are underlined, with mutations resulting in amino acid substitutions in the putative Gm-moricinA precursor protein indicated by a double underline.

FIG. 2. Sequence alignment of the deduced protein sequences of two Gm-moricinA cDNA clones (GmmoriAe—SEQ ID NO:1; GmmoriAc—SEQ ID NO:2). Non conserved residues in clone GmmoriAc are underlined.

FIG. 3. Nucleotide sequence and deduced pre-pro protein sequence of the G. mellonella Gm-moricinA cDNA clone pGmmoriAa (SEQ ID NO's 29 and 1 respectively). The deduced protein sequence commences at the first in-frame methionine residue. The presumptive secretion signal peptide is shown in italics and the mature Gm-moricinA peptide is highlighted in bold font. The peptide sequence obtained by Edman sequencing of the purified Gm-moricinA peptide is shown underlined. The predicted site of signal peptide cleavage (SignalP) is indicated below the peptide sequence by a single arrow and the predicted site of cleavage to generate the mature form of the peptide is indicated by a pair of arrows.

FIG. 4. Nucleotide sequence and deduced pre-pro protein sequence of the G. mellonella Gm-moricinB cDNA (SEQ ID NO's 8 and 3 respectively). The deduced protein sequence commences at the first in-frame methionine residue. The presumptive secretion signal peptide is shown in italics and the mature Gm-moricinB peptide is highlighted in bold font. The peptide sequence obtained by Edman sequencing of the purified Gm-moricinB peptide is shown underlined. The predicted site of signal peptide cleavage (SignalP) is indicated below the peptide sequence by a single arrow and the predicted site of cleavage to generate the mature form of the peptide is indicated by a pair of arrows.

FIG. 5. Nucleotide sequence and deduced pre-pro protein sequence of the G. mellonella Gm-moricinC1 cDNA clone Gm3-01ae (SEQ ID NO's 49 and 47 respectively). The deduced protein sequence commences at the first in-frame methionine residue. The presumptive secretion signal peptide is shown in italics and the mature Gm-moricinC1 peptide is highlighted in bold font. The peptide sequence obtained by Edman sequencing of the purified Gm-moricinC1 peptide is shown underlined. The predicted site of signal peptide cleavage (SignalP) is indicated below the peptide sequence by a single arrow and the predicted site of cleavage to generate the mature form of the peptide is indicated by a pair of arrows. The three non-conserved nucleotides are underlined, with the mutation in the open reading frame which results in an amino acid substitution indicated by a double underline. The single amino acid change resulting from this nucleotide substitution is shown below the amino acid sequence in italics.

FIG. 6. Nucleotide sequence and deduced pre-pro protein sequence of the G. mellonella Gm-moricinC2 cDNA clone Gm3-03 (SEQ ID NO's 54 and 52 respectively). The deduced protein sequence commences at the first in-frame methionine residue. The presumptive secretion signal peptide is shown in italics and the mature Gm-moricinC2 peptide is highlighted in bold font. The predicted site of signal peptide cleavage (SignalP) is indicated below the peptide sequence by a single arrow and the predicted site of cleavage to generate the mature form of the peptide is indicated by a pair of arrows. A possible site for the polyA signal is indicated by a dotted underline.

FIG. 7. Sequence alignment of the nucleotide sequences of the cDNA clones of Gm-moricinC1 (Gm3-01ae—SEQ ID NO:49) and Gm-moricinC2 (Gm3-03,—SEQ ID NO:54). The start and stop codons are shown in bold. The 19 nucleotides in the open reading frame of Gm-moricinC2 that differ to Gm-moricinC1 are underlined, with mutations resulting in amino acid substitutions indicated by a double underline.

FIG. 8. Sequence alignment of the deduced protein sequences of Gm-moricinC1 (SEQ ID NO:47) and Gm-moricinC2 (SEQ ID NO:52). Non-conserved residues are underlined. Note that an allelic variant of Gm-moricinC1 was found with a VAL residue at position 13.

FIG. 9. ClustalW alignment of the antifungal peptides from G. mellonella (GmmoriA—SEQ ID NO:1, GmmoriB—SEQ ID NO:3, GmmoriC1—SEQ ID NO:47 and GmmoriC2—SEQ ID NO:52) with related peptides from the Lepidoptera Bombyx mori (Bmmor, P82818) (SEQ ID NO:16), Spodoptera litura (Slmor, BAC79440) (SEQ ID NO:14), Spodoptera exigua (Semor, AAT38873) (SEQ ID NO:57), Manduca sexta (Msmor, AAO74637) (SEQ ID NO:15), Heliothis virescens (Hvvir, P83416) (SEQ ID NO:17), Hyblaea puera (Hpmor, AAW21268) (SEQ ID NO:58), Caligo illioneus (CiP1646, CiP1647, CiP1648—SEQ ID NO's 59, 60 and 61 respectively). Also included in the alignment is a previously unannotated putative moricin from Bombyx mori (BmmorX, BP125548) (SEQ ID NO:18). The G. mellonella sequences correspond to the translated open reading frame from the cDNA sequences.

### KEY TO THE SEQUENCE LISTING

SEQ ID NO:1—Pre-Gm-moricinA from Galleria mellonella.

SEQ ID NO:2—Allelic variant (GmmoriAc) of pre-Gm-moricinA from Galleria mellonella.

SEQ ID NO:3—Pre-Gm-moricinB from Galleria mellonella.

SEQ ID NO:4—Gm-moricinA from Galleria mellonella.

SEQ ID NO:5—Gm-moricinB from Galleria mellonella.

SEQ ID NO:6—cDNA encoding pre-Gm-moricinA including known 5′ and 3′ untranslated sequence from Galleria mellonella.

SEQ ID NO:7—cDNA encoding allelic variant (GmmoriAc) of pre-Gm-moricinA including known 5′ and 3′ untranslated sequence from Galleria mellonella.

SEQ ID NO:8—cDNA encoding pre-Gm-moricinB including known 5′ and 3′ untranslated sequence from Galleria mellonella.

SEQ ID NO:9—cDNA encoding pre-Gm-moricinA from Galleria mellonella.

SEQ ID NO:10—cDNA encoding allelic variant (GmmoriAc) of pre-Gm-moricinA from Galleria mellonella.

SEQ ID NO:11—cDNA encoding pre-Gm-moricinB from Galleria mellonella.

SEQ ID NO:12—cDNA encoding Gm-moricinA from Galleria mellonella.

SEQ ID NO:13—cDNA encoding Gm-moricinB from Galleria mellonella.

SEQ ID NO:14—Moricin-like pre-peptide from Spodoptera litura (predicted peptide from Genbank Accession No. BAC79440).

SEQ ID NO:15—Moricin-like pre-peptide from Manduca sexta (predicted peptide from Genbank Accession No. AAO74637).

SEQ ID NO:16—Pre-moricin from Bombyx mori (Hara and Yamakawa (1995) and Genbank Accession No. P82818).

SEQ ID NO:17—Virescein (moricin-like peptide) from Heliothis virescens (Genbank Accession No. P83416).

SEQ ID NO:18—Bombyx mori moricin-X (encoded by Genbank Accession BP125548).

SEQ ID NO:19—N-terminal sequence of isolated Gm-moricinA.

SEQ ID NO:20—N-terminal sequence of isolated Gm-moricinB.

SEQ ID NO's:21 to 28—Oligonucleotide primers.

SEQ ID NO:29—Polynucleotide sequence of clone GmmoriAa.

SEQ ID NO:30—N-terminal sequence of isolated Gm-moricinC1.

SEQ ID NO's:31 to 46—Oligonucleotide primers.

SEQ ID NO:47—Pre-Gm-moricinC1 from Galleria mellonella.

SEQ ID NO:48—Gm-moricinC1 from Galleria mellonella.

SEQ ID NO:49—cDNA encoding pre-Gm-moricinC1 including known 5′ and 3′ untranslated sequence from Galleria mellonella.

SEQ ID NO:50—cDNA encoding pre-Gm-moricinC1 from Galleria mellonella.

SEQ ID NO:51—cDNA encoding Gm-moricinC1 from Galleria mellonella.

SEQ ID NO:52—Pre-Gm-moricinC2 from Galleria mellonella.

SEQ ID NO:53—Gm-moricinC2 from Galleria mellonella.

SEQ ID NO:54—cDNA encoding pre-Gm-moricinC2 including known 5′ and 3′ untranslated sequence from Galleria mellonella.

SEQ ID NO:55—cDNA encoding pre-Gm-moricinC2 from Galleria mellonella.

SEQ ID NO:56—cDNA encoding Gm-moricinC2 from Galleria mellonella.

SEQ ID NO:57—Moricin-like peptide from Spodoptera exigua (predicted pre-peptide from Genbank Accession No. AAT38873).

SEQ ID NO:58—Moricin-like peptide from Hyblaea puera (predicted pre-peptide from Genbank Accession No. AAW21268).

SEQ ID NO:59—Moricin-like peptide from Caligo illioneus (CiP1646, described in WO 2004/016650).

SEQ ID NO:60—Moricin-like peptide from Caligo illioneus (CiP1647, described in WO 2004/016650).

SEQ ID NO:61—Moricin-like peptide from Caligo illioneus (CiP1648, described in WO 2004/016650).

SEQ ID NO:62—Consensus sequence for Galleria antifungal peptides.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, microbiology, molecular genetics, immunology, immunohistochemistry, protein chemistry, mycology and biochemistry).

Unless otherwise indicated, the recombinant protein, cell culture, transgenic plant production and microbiological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), and are incorporated herein by reference.

As used herein, the term “antifungal” peptide refers to a peptide having antifungal properties, e.g., which inhibits the growth of fungal cells, or which kills fungal cells, or which disrupts or retards stages of the fungal life cycle such as spore germination, sporulation, and mating.

As used herein, the term “antibacterial” peptide refers to a peptide having antibacterial properties, e.g., which inhibits the growth of bacterial cells, or which kills bacterial cells, or which disrupts or retards stages of the bacteria life cycle such as spore formation, and cell division.

### Polypeptides/Peptides

By “substantially purified peptide” we mean a peptide that has generally been separated from the lipids, nucleic acids, other peptides, and other contaminating molecules with which it is associated in its native state. Preferably, the substantially purified peptide is at least 60% free, more preferably at least 75% free, and more preferably at least 90% free from other components with which it is naturally associated.

The terms “polypeptide” and “peptide” are generally used interchangeably. However, the term “peptide” is typically used to refer to chains of amino acids which are not large, for instance 100 or less residues in length.

The % identity of a peptide is determined by GAP (Needleman and Wunsch, 1970) analysis (GCG program) with a gap creation penalty=8, and a gap extension penalty=3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids.

As used herein a “biologically active” fragment is a portion of a peptide of the invention which maintains a defined activity of the full length peptide. In most embodiments this activity is antifungal activity, however, in some embodiments this activity is antibacterial. Biologically active fragments can be any size as long as they maintain the defined activity, however, in a preferred embodiment they are at least 10, more preferably at least 15, amino acids in length.

Amino acid sequence mutants of the peptides of the present invention, can be prepared by introducing appropriate nucleotide changes into a nucleic acid of the present invention, or by in vitro synthesis of the desired peptide. Such mutants include, for example, deletions, insertions or substitutions of residues within the amino acid sequence. A combination of deletion, insertion and substitution can be made to arrive at the final construct, provided that the final peptide product possesses the desired characteristics.

Mutant (altered) peptides can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). These DNA shuffling techniques may include genes related to those of the present invention, such as that encoding moricin from B. mori (Hara and Yamakawa, 1995). Peptide products derived from mutated/altered DNA can readily be screened using techniques described herein to determine if they possess antifungal and/or antibacterial activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the peptide molecule removed and a different residue inserted in its place. The sites of greatest interest for substitutional mutagenesis include sites identified as the active site(s). Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

TABLE 1
Exemplary substitutions

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

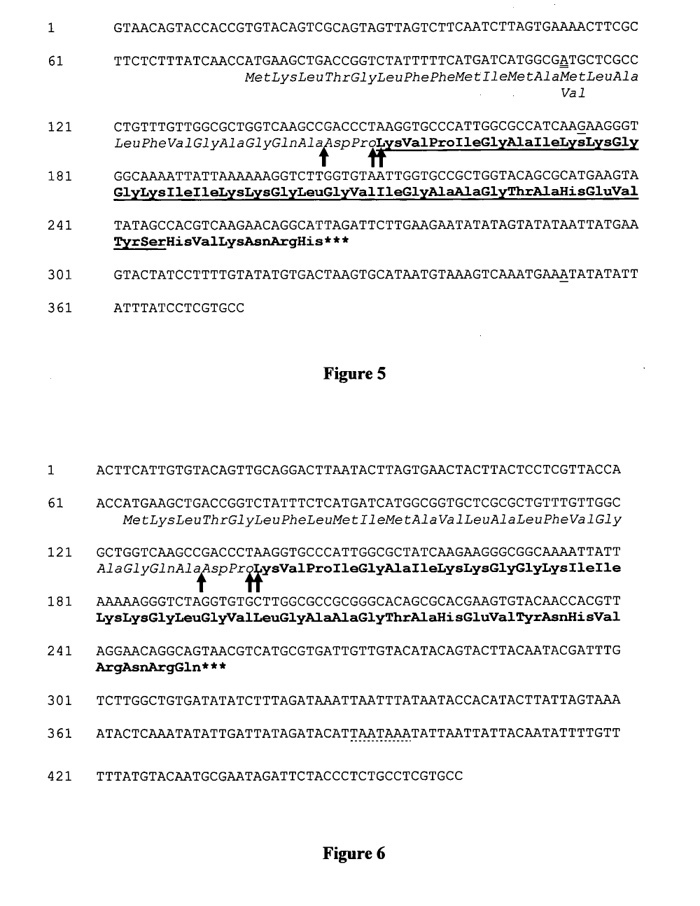

In particular, it has previously been shown that moricin possesses two α-helical structures (Hemmi et al, 2002). Considering the relatedness of the peptides of the invention to moricin-like peptides (see FIG. 5), it is possible that a similar structure is also important for maintaining antifungal activity of the peptides of the invention. Accordingly, when designing mutants of, for example, SEQ ID NO:4 the skilled addressee, using knowledge of the chemistry of particular amino acids combined with known methods of predicting peptide tertiary structure, can readily produce peptides with one or a few amino acid variations when compared to SEQ ID NO:4 which possess antifungal activity.

Furthermore, if desired, unnatural amino acids or chemical amino acid analogues can be introduced as a substitution or addition into the peptides of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are peptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the peptide of the invention.

Peptides of the present invention can be produced in a variety of ways, including production and recovery of natural peptides, production and recovery of recombinant peptides, and chemical synthesis of the peptides. In one embodiment, an isolated peptide of the present invention is produced by culturing a cell capable of expressing the peptide under conditions effective to produce the peptide, and recovering the peptide. A preferred cell to culture is a recombinant cell of the present invention. Effective culture conditions include, but are not limited to, effective media, bioreactor, temperature, pH and oxygen conditions that permit peptide production. An effective medium refers to any medium in which a cell is cultured to produce a peptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Polynucleotides

By “isolated polynucleotide” we mean a polynucleotide which has generally been separated from the polynucleotide sequences with which it is associated or linked in its native state. Preferably, the isolated polynucleotide is at least 60% free, more preferably at least 75% free, and more preferably at least 90% free from other components with which it is naturally associated. Furthermore, the term “polynucleotide” is used interchangeably herein with the term “nucleic acid molecule”.

The % identity of a polynucleotide is determined by GAP (Needleman and Wunsch, 1970) analysis (GCG program) with a gap creation penalty=8, and a gap extension penalty=3. The query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. Even more preferably, the query sequence is at least 300 nucleotides in length and the GAP analysis aligns the two sequences over a region of at least 300 nucleotides.

A polynucleotide of the present invention may selectively hybridise to a polynucleotide that encodes a peptide of the present invention under high stringency. Furthermore, oligonucleotides of the present invention have a sequence that hybridizes selectively under high stringency to a polynucleotide of the present invention. As used herein, high stringency conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis or DNA shuffling on the nucleic acid as described above). It is thus apparent that polynucleotides of the invention can be either naturally occurring or recombinant.

Oligonucleotides of the present invention can be RNA, DNA, or derivatives of either. The minimum size of such oligonucleotides is the size required for the formation of a stable hybrid between an oligonucleotide and a complementary sequence on a nucleic acid molecule of the present invention. The present invention includes oligonucleotides that can be used as, for example, probes to identify nucleic acid molecules, or primers to amplify nucleic acid molecules of the invention.

### Recombinant Vectors

One embodiment of the present invention includes a recombinant vector, which comprises at least one isolated polynucleotide molecule of the present invention, inserted into any vector capable of delivering the polynucleotide molecule into a host cell. Such a vector contains heterologous polynucleotide sequences, that is polynucleotide sequences that are not naturally found adjacent to polynucleotide molecules of the present invention and that preferably are derived from a species other than the species from which the polynucleotide molecule(s) are derived. The vector can be either RNA or DNA, either prokaryotic or eukaryotic, and typically is a transposon (such as described in U.S. Pat. No. 5,792,294), a virus or a plasmid.

One type of recombinant vector comprises a polynucleotide molecule of the present invention operatively linked to an expression vector. The phrase operatively linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, animal, and plant cells. Particularly preferred expression vectors of the present invention can direct gene expression in plants cells. Vectors of the invention can also be used to produce the peptide in a cell-free expression system, such systems are well known in the art.

In particular, expression vectors of the present invention contain regulatory sequences such as transcription control sequences, translation control sequences, origins of replication, and other regulatory sequences that are compatible with the recombinant cell and that control the expression of polynucleotide molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod and mammalian cells, such as, but not limited to, tac, lac, trp, trc, oxy-pro, omp/lpp, rrnB, bacteriophage lambda, bacteriophage T7, T71ac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Particularly preferred transcription control sequences are promoters active in directing transcription in plants, either constitutively or stage and/or tissue specific, depending on the use of the plant or parts thereof. These plant promoters include, but are not limited to, promoters showing constitutive expression, such as the 35S promoter of Cauliflower Mosaic Virus (CaMV), those for leaf-specific expression, such as the promoter of the ribulose bisphosphate carboxylase small subunit gene, those for root-specific expression, such as the promoter from the glutamine synthase gene, those for seed-specific expression, such as the cruciferin A promoter from Brassica napus, those for tuber-specific expression, such as the class-I patatin promoter from potato or those for fruit-specific expression, such as the polygalacturonase (PG) promoter from tomato.

Recombinant molecules of the present invention may also (a) contain secretory signals (i.e., signal segment nucleic acid sequences) to enable an expressed peptide of the present invention to be secreted from the cell that produces the peptide and/or (b) contain fusion sequences which lead to the expression of nucleic acid molecules of the present invention as fusion proteins. Examples of suitable signal segments include any signal segment capable of directing the secretion of a peptide of the present invention. Preferred signal segments include, but are not limited to, tissue plasminogen activator (t-PA), interferon, interleukin, growth hormone, viral envelope glycoprotein signal segments, Nicotiana nectarin signal peptide (U.S. Pat. No. 5,939,288), tobacco extensin signal, the soy oleosin oil body binding protein signal, Arabidopsis thaliana vacuolar basic chitinase signal peptide, as well as native signal sequences of the peptide of the invention. In addition, a nucleic acid molecule of the present invention can be joined to a fusion segment that directs the encoded peptide to the proteosome, such as a ubiquitin fusion segment. Recombinant molecules may also include intervening and/or untranslated sequences surrounding and/or within the nucleic acid sequences of nucleic acid molecules of the present invention.

### Host Cells

Another embodiment of the present invention includes a recombinant cell comprising a host cell transformed with one or more recombinant molecules of the present invention. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotide molecules of the present invention can remain extrachromosomal or can integrate into one or more sites within a chromosome of the transformed (i.e., recombinant) cell in such a manner that their ability to be expressed is retained.

Although peptides discussed herein possess antifungal and antibacterial activity, suitable quantities of recombinant peptide of the invention can be obtained from bacterial or fungal host cells. More specifically, the peptide can be produced as a fusion protein, which is processed upon recovering the fusion protein from the recombinant host cell. An example of such a system is described by Hara and Yamakawa (1996) whereby moricin (SEQ ID NO:16) was produced as a fusion protein from E. coli. The fusion protein was harvested from the recombinant host cells and cleaved with cyanogen or o-iodosobenzoic acid to release the bioactive moricin peptide. A similar system could readily be devised to produce peptides of the present invention in bacterial or fungal host cells.

Suitable host cells to transform include any cell that can be transformed with a polynucleotide of the present invention. Host cells of the present invention either can be endogenously (i.e., naturally) capable of producing peptides of the present invention or can be capable of producing such peptides after being transformed with at least one polynucleotide molecule of the present invention. Host cells of the present invention can be any cell capable of producing at least one protein of the present invention, and include bacterial, fungal (including yeast), parasite, arthropod, animal and plant cells. Examples of host cells include Salmonella, Escherichia, Bacillus, Listeria, Saccharomyces, Spodoptera, Mycobacteria, Trichoplusia, BHK (baby hamster kidney) 35 cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; BHK cells; MDCK cells; CRFK cells; CV-1 cells; COS cells; Vero cells; and non-tumorigenic mouse myoblast G8 cells (e.g., ATCC CRL 1246). Additional appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK cells and/or HeLa cells. Particularly preferred host cells are plant cells such as those available from Deutsche Sammlung von Mikroorganismen und Zelikulturen GmbH (German Collection of Microorganisms and Cell Cultures).

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules of the present invention include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts.

### Transgenic Plants

The term “plant” refers to whole plants, plant organs (e.g. leaves, stems roots, etc), seeds, plant cells and the like. Plants contemplated for use in the practice of the present invention include both monocotyledons and dicotyledons. Preferably, the transgenic plant is a commercially useful crop plant. Target crops include, but are not limited to, the following: cereals (wheat, barley, rye, oats, rice, sorghum and related crops); beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (rape, mustard, poppy, olives, sunflowers, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf, bananas and natural rubber plants, as well as ornamentals (flowers, shrubs, broad-leaved trees and evergreens, such as conifers). Particularly preferred crops include field peas, chickpeas, wheat and barley.

Transgenic plants, as defined in the context of the present invention include plants (as well as parts and cells of said plants) and their progeny which have been genetically modified using recombinant techniques to cause production of at least one peptide of the present invention in the desired plant or plant organ. Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

A polynucleotide of the present invention may be expressed constitutively in the transgenic plants during all stages of development. Depending on the use of the plant or plant organs, the peptides may be expressed in a stage-specific manner. Furthermore, depending on the use—particularly where the plant may be prone to fungal infection, the polynucleotides may be expressed tissue-specifically.

Regulatory sequences which are known or are found to cause expression of a gene encoding a peptide of interest in plants may be used in the present invention. The choice of the regulatory sequences used depends on the target plant and/or target organ of interest. Such regulatory sequences may be obtained from plants or plant viruses, or may be chemically synthesized. Such regulatory sequences are well known to those skilled in the art.

Other regulatory sequences such as terminator sequences and polyadenylation signals include any such sequence functioning as such in plants, the choice of which would be obvious to the skilled addressee. An example of such sequences is the 3′ flanking region of the nopaline synthase (nos) gene of Agrobacterium tumefaciens.

Several techniques are available for the introduction of an expression construct containing a nucleic acid sequence encoding a peptide of interest into the target plants. Such techniques include but are not limited to transformation of protoplasts using the calcium/polyethylene glycol method, electroporation and microinjection or (coated) particle bombardment. In addition to these so-called direct DNA transformation methods, transformation systems involving vectors are widely available, such as viral and bacterial vectors (e.g. from the genus Agrobacterium). After selection and/or screening, the protoplasts, cells or plant parts that have been transformed can be regenerated into whole plants, using methods known in the art. The choice of the transformation and/or regeneration techniques is not critical for this invention.

Examples of transgenic plants expressing antifungal peptides are described in Banzet et al. (2002) and EP 798381. In each case, the expression of the recombinant antifungal peptide resulted in the transgenic plant being resistant to fungal infections. Similar procedures as outlined in these documents can be used to produce peptides of the invention which confer resistance to fungal infections to the transgenic plant.

### Transgenic Hon-Human Animals

Techniques for producing transgenic animals are well known in the art. A useful general textbook on this subject is Houdebine, Transgenic animals—Generation and Use (Harwood Academic, 1997).

Heterologous DNA can be introduced, for example, into fertilized mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals.

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology. Using this method, fibroblasts from donor animals are stably transfected with a plasmid incorporating the coding sequences for a binding domain or binding partner of interest under the control of regulatory sequences. Stable transfectants are then fused to enucleated oocytes, cultured and transferred into female recipients.

### Compositions

Compositions of the present invention include “acceptable carriers”. An acceptable carrier is preferably any material that the animal, plant, plant or animal material, or environment (including soil and water samples) to be treated can tolerate. Examples of such acceptable carriers include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used.

Pharmaceutical compositions contain a therapeutically effective amount of an antifungal peptide of the invention. A therapeutically effective amount of an antifungal peptide can be readily determined according to methods known in the art. Pharmaceutical compositions are formulated to contain the therapeutically effective amount of an antifungal peptide and a pharmaceutically acceptable carrier appropriate for the route of administration (topical, gingival, intravenous, aerosol, local injection) as known to the art. For agricultural use, the composition comprises a therapeutically effective amount of a peptide of the invention and an agriculturally acceptable carrier suitable for the organism (e.g., plant) to be treated.

The phrase ‘pharmaceutically acceptable carrier’ refers to molecular entities and compositions that do not produce an allergic, toxic or otherwise adverse reaction when administered to an animal, particularly a mammal, and more particularly a human. Useful examples of pharmaceutically acceptable carriers or diluents include, but are not limited to, solvents, dispersion media, coatings, stabilizers, protective colloids, adhesives, thickeners, thixotropic agents, penetration agents, sequestering agents and isotonic and absorption delaying agents that do not affect the activity of the peptides of the invention. The proper fluidity can be maintained, for example, by the use of a coating, such as lecithin, by the maintenance of the required particle size in the case of dispersion and by the use of surfactants. More generally, the peptides of the invention can be combined with any non-toxic solid or liquid additive corresponding to the usual formulating techniques.

Liquid compositions of the invention include water-soluble concentrates, emulsifiable concentrates, emulsions, concentrated suspensions, aerosols, wettable powders (or powder for spraying), pastes and gels.

A peptide of the invention can also be used in the form of powders for dusting, and granules, in particular those obtained by extrusion, compacting, impregnation of a granular carrier or by granulation of a powder, and effervescent tablets or lozenges.

Surfactants may also form a component of various compositions. Surfactants can be an emulsifier, dispersant or wetting agent of ionic or nonionic type or a mixture of such surfactants. Examples include, but are not limited to, polyacrylic acid salts, lignosulfonic acid salts, phenolsulfonic or naphthalenesulfonic acid salts, polycondensates of ethylene oxide with fatty alcohols or with fatty acids or with fatty amines, substituted phenols (in particular alkyophenols or arylphenols), salts of sulfosuccinic acid esters, taurine derivatives (in particular alkyl taurates), polyoxyethylated phosphoric esters of alcohols or of phenols, fatty acid esters of polyols, derivatives containing sulfate, sulfonate and phosphate functions of the above compounds.

Depending on the specific conditions being treated and the targeting method selected, such agents may be formulated and administered systemically or locally. Suitable routes may include, for example, oral, rectal, transdermal, vaginal, transmucosal, or intestinal administration; parenteral delivery, including intramuscular, subcutaneous, or intramedullary injections, as well as intrathecal, intravenous, or intraperitoneal injections.

For agricultural compositions, natural or synthetic, organic or inorganic materials may be used with which the compound is combined in order to facilitate its application to the plant, to seeds or the soil. This carrier is thus generally inert and it should be agriculturally acceptable, in particular on the plant treated. The carrier can be solid (clays, natural or synthetic silicates, silica, resins, waxes, solid fertilizers, etc.) or liquid (water, alcohols, in particular butanol, etc.).

Exposure of a plant pathogen to an antifungal peptide may be achieved by applying to plant parts or to the soil or other growth medium surrounding the roots of the plants or to the seed of the plant before it is sown using standard agricultural techniques such as spraying. The peptide may be applied to plants or to the plant growth medium in the form of a composition comprising the peptide in admixture with a solid or liquid diluent and optionally various adjuvants such as surface-active agents. Solid compositions may be in the form of dispersible powders, granules, or grains.

The compositions of the present invention can also be used in numerous products including, but not limited to, disinfectant hand soaps, hypo-allergenic hand care creme, shampoo, face soap, laundry products, dish washing products (including a bar glass dip) bathroom cleaning products, dental products (e.g., mouthwash, dental adhesive, saliva injector filters, water filtration) and deodorizing products.

One embodiment of the present invention is a controlled release formulation that is capable of slowly releasing a peptide of the present invention into an animal, plant, animal or plant material, or the environment (including soil and water samples). As used herein, a controlled release formulation comprises a peptide of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Preferred controlled release formulations are biodegradable (i.e., bioerodible).

The formulation is preferably released over a period of time ranging from about 1 to about 12 months. A preferred controlled release formulation of the present invention is capable of effecting a treatment preferably for at least about 1 month, more preferably for at least about 3 months, even more preferably for at least about 6 months, even more preferably for at least about 9 months, and even more preferably for at least about 12 months.

The effective concentration of the peptide, vector, or host cell within the composition can readily be determined experimentally, as will be understood by the skilled artisan.

Examples of compositions comprising antifungal peptides is provided in U.S. Pat. No. 6,331,522. Similar compositions comprising the peptides of the invention could readily be produced by the skilled addressee.

### Antibodies

The invention also provides monoclonal or polyclonal antibodies to peptides of the invention or fragments thereof. Thus, the present invention further provides a process for the production of monoclonal or polyclonal antibodies to peptides of the invention.

The term “binds specifically” refers to the ability of the antibody to bind to at least one protein/peptide of the present invention but not other known moricin-like peptides such as those provided in SEQ ID NO's 14 to 17.

As used herein, the term “epitope” refers to a region of a peptide of the invention which is bound by the antibody. An epitope can be administered to an animal to generate antibodies against the epitope, however, antibodies of the present invention preferably specifically bind the epitope region in the context of the entire peptide.

If polyclonal antibodies are desired, a selected mammal (e.g., mouse, rabbit, goat, horse, etc.) is immunised with an immunogenic peptide. Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides peptides of the invention or fragments thereof haptenised to another peptide for use as immunogens in animals.

Monoclonal antibodies directed against peptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies of the invention may be bound to a solid support and/or packaged into kits in a suitable container along with suitable reagents, controls, instructions and the like.

Preferably, antibodies of the present invention are detectably labeled. Exemplary detectable labels that allow for direct measurement of antibody binding include radiolabels, fluorophores, dyes, magnetic beads, chemiluminescers, colloidal particles, and the like. Examples of labels which permit indirect measurement of binding include enzymes where the substrate may provide for a coloured or fluorescent product. Additional exemplary detectable labels include covalently bound enzymes capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Further exemplary detectable labels include biotin, which binds with high affinity to avidin or streptavidin; fluorochromes (e.g., phycobiliproteins, phycoerythrin and allophycocyanins; fluorescein and Texas red), which can be used with a fluorescence activated cell sorter; haptens; and the like. Preferably, the detectable label allows for direct measurement in a plate luminometer, e.g., biotin. Such labeled antibodies can be used in techniques known in the art to detect peptides of the invention.

### Uses

The peptides of the invention have many uses in medical, veterinary, agricultural, food preservative, household and industrial areas where it is desirable to reduce and/or prevent fungal or bacterial infections.

For instance, the peptides of the present invention can be used in pharmaceutical compositions to treat fungal infections, as well as bacterial infections (e.g., S. mutans, P aeruginosa or P. gingivalis infections). Vaginal, urethral, mucosal, respiratory, skin, ear, oral, or ophthalmic fungal or bacterial infections that are amenable to peptide therapy include, but are not limited to: Candida albicans; Actinomyces actinomycetemcomitans; Actinomyces viscosus; Bacteriodesforsythus; Bacteriodesfragilis; Bacteriodes gracilis; Bacteriodes ureolyticus; Campylobacter concisus; Campylobacter rectus; Campylobacter showae; Campylobacter sputorum; Capnocytophaga gingivalis; Capnocytophaga ochracea; Capnocytophaga sputigena; Clostridium histolyticum; Eikenella corrodens; Eubacterium nodatum; Fusobacterium nucleatum; Fusobacterium periodonticum; Peptostreptococcus micros; Porphyromonas endodontalis; Porphyromonas gingivalis; Prevotella intermedia; Prevotella nigrescens; Propionibacterium acnes; Pseudomonas aeruginosa; Selenomonas noxia; Staphylococcus aureus; Streptococcus constellatus; Streptococcus gordonii; Streptococcus intermedius; Streptococcus mutans; Streptococcus oralis; Streptococcus pneumonia; Streptococcus sanguis; Treponema denticola; Treponema pectinovorum; Treponema socranskii; Veillonellaparvula; and Wolinella succinogenes.

For agricultural applications, the antifungal peptide may be used to improve the disease-resistance or disease-tolerance of crops either during the life of the plant or for post-harvest crop protection. The growth of pathogens exposed to the peptides is inhibited. The antifungal peptide may eradicate a pathogen already established on the plant or may protect the plant from future pathogen attack. A pathogen may be any fungus growing on, in or near the plant. Improved resistance is defined as enhanced tolerance of the plant, or the crop after harvesting, to a fungal pathogen when compared to a wild-type plant. Resistance may vary from a slight decrease in the effects, to the total eradication so that the plant is unaffected by the presence of pathogen.

Thus, peptides of the invention can also be used to treat and/or prevent fungal infections of plants. Such plant fungi include, but are not limited to, those selected from the group consisting of the Genera: Alternaria; Ascochyta; Botrytis; Cercospora; Colletotrichum; Diplodia; Erysiphe; Fusarium; Leptosphaeria; Gaeumanomyces; Helminthosporium; Macrophomina; Nectria; Peronospora; Phoma; Phymatotrichum; Phytophthora; Plasmopara; Podosphaera; Puccinia; Puthium; Pyrenophora; Pyricularia; Pythium; Rhizoctonia; Scerotium; Sclerotinia; Septoria; Thielaviopsis; Uncinula; Venturia; and Verticillium. Specific examples of plant fungi infections which may be treated with the peptides of the present invention include, Erysiphe graminis in cereals, Erysiphe cichoracearum and Sphaerotheca fuliginea in cucurbits, Podosphaera leucotricha in apples, Uncinula necator in vines, Puccinia sp. in cereals, Rhizoctonia sp. in cotton, potatoes, rice and lawns, Ustilago sp. in cereals and sugarcane, Venturia inaequalis (scab) in apples, Helminthosporium sp. in cereals, Septoria nodorum in wheat, Septoria tritici in wheat, Rhynchosporium secalis on barley, Botrytis cinerea (gray mold) in strawberries, tomatoes and grapes, Cercospora arachidicola in groundnuts, Peronospora tabacina in tobacco, or other Peronospora in various crops, Pseudocercosporella herpotrichoides in wheat and barley, Pyrenophera teres in barley, Pyricularia oryzae in rice, Phytophthora infestans in potatoes and tomatoes, Fusarium sp. (such as Fusarium oxysporum) and Verticillium sp. in various plants, Plasmopara viticola in grapes, Alternaria sp. in fruit and vegetables, Pseudoperonospora cubensis in cucumbers, Mycosphaerella fijiensis in banana, Ascochyta sp. in chickpeas, Leptosphaeria sp. on canola, and Colleotrichum sp. in various crops.

An antifungal peptide according to the invention may also be used as a preservative to maintain the freshness and shelf life of food products such as cheese, bread, cakes, meat, fish, preserves, feed for animals and the like. The antifungal peptide may also be used in antimicrobial food packaging such as coating plastics or polymers or incorporation within edible coating or films. For example peptide coatings and films can contain adequate amounts of antifungal peptide(s) for use on such products as cheese, sweets, dried goods and the like.

### EXAMPLES

### Example 1

### Peptide Purification

### Materials and Methods

### Insects

Galleria mellonella (wax moth) were reared on an artificial diet. Last instar larvae were injected with 10 μl of water containing approximately 10⁶ cells of each of E. coli and M. luteus. As a control, some larvae were injected with 10 μL of phosphate buffered saline solution. Larvae were left at room temperature for 48 hours before extracting hemolymph by removal of a proleg. Hemolymph was collected on ice in a tube containing a few crystals of phenylthiourea, centrifuged for 5 min to remove cell debris, and frozen at −80° C.

### Antifungal and Antibacterial Activity Assays

Samples were tested for activity using an inhibition zone plate assay. For the bacteria Escherichia coli and Micrococcus luteus, plates were prepared using nutrient agar (Oxoid) and a cell density of approximately 5×10⁶ cells/ml.

For fungi, plates were prepared using YPD broth (10 g/l yeast extract, 10 g/l peptone, 40 g/l D-glucose) containing 0.8% agarose and a spore density of approximately 10⁶ spores/ml. To test for activity, 2 μl of the sample of interest was spotted on the surface of the plate, and the organism grown under appropriate conditions (overnight at 37° C. for bacteria, 1-3 days at room temperature for fungi) until the presence or absence of clearance zones could be detected. The fungi tested were Fusarium graminearum, Alternaria alternata, Ascochyta rabiei, Colletotrichum gloeosporioides, Leptosphaeria maculans and Aspergillus niger.

### Peptide Purification

Two crude hemolymph samples from different G. mellonella immunisations were processed by C18 solid phase extraction. The thawed hemolymph (1.4 or 4.8 ml) was diluted into an equal volume of 0.1% trifluoroacetic acid (TFA), and shaken on ice for 30-45 min. The samples were centrifuged at high speed for 10 min and the supernatant removed. The first sample (from 1.4 ml of hemolymph) was precipitated with 20% acetonitrile/0.05% TFA, and re-centrifuged for 5 min at high speed. The supernatant was loaded onto three C18 solid phase extraction cartridges (Maxi-Clean, 300 mg cartridges, Alltech) equilibrated in 20% acetonitrile/0.05% TFA. Each cartridge was washed with 20% acetonitrile/0.05% TFA, and eluted with 1 ml of 60% acetonitrile/0.05% TFA. The second sample (from 4.8 ml of hemolymph) was loaded onto three C18 solid phase extraction cartridges (Maxi-Clean, 900 mg cartridges, Alltech) equilibrated in 0.05% TFA. The cartridges were washed with 0.05% TFA and eluted stepwise with 3 ml 20% acetonitrile/0.05% TFA followed by 3 ml 60% acetonitrile/0.05% TFA. Samples (1 ml) from the 60% acetonitrile/0.05% TFA elution were dried in a Speedvac (Savant) and resuspended in 100 μl water. The samples were tested against E. coli, M. luteus and various fungi using the plate assay described above.

The crude hemolymph sample was purified by reverse phase HPLC on a Beckman Gold system monitoring absorbance at 225 or 215 nm. Sample (1.4-1.8 ml) was loaded onto a Jupiter C18, 5 μm, 300 Å, 250×10 mm semi-prep column (Phenomenex) equilibrated in solvent A (2% acetonitrile, 0.065% TFA), and eluted with a gradient from 0-70% solvent B (95% acetonitrile, 0.05% TFA) over 70 min at 5 ml/min. 500 μl of each 5 ml fraction was dried in the Speedvac, resuspended in 10 μl water, and tested for activity against E. coli, M. luteus and F. graminearum, as described above.

For Gm-moricinA, the fraction of interest was diluted in an equal volume of 0.05% TFA and loaded onto a Prosphere C18, 5 μm, 300 Å, 250×4.6 mm analytical column (Alltech) equilibrated in 10% solvent B on the HPLC. The column was eluted with a gradient of 10-50% B running over 60 min at 1 ml/min. 200 μl of each 1.8 ml fraction was dried in the Speedvac, resuspended in 10 μl water and tested for activity against E. coli, M. luteus and F. graminearum. The fraction of interest was diluted in an equal volume of 0.05% TFA and loaded onto a Macrosphere C8, 5 μm, 300 Å, 250×4.6 mm analytical column (Alltech) equilibrated in 15% solvent B on the HPLC. The column was eluted with a gradient of 15-55% solvent B running over 60 min at 1 ml/min. 300 μl of each 1.8 ml fraction was dried in the Speedvac, resuspended in 10 μl water and tested for activity against E. coli, M. luteus and F. graminearum.

For Gm-moricinB, the fraction of interest was diluted in an equal volume of 0.05% TFA and loaded onto a Prosphere C18, 5 μm, 300 Å, 250×4.6 mm analytical column (Alltech) equilibrated in 15% solvent B on the HPLC. The column was eluted with a gradient of 15-65% B running over 75 min at 1 ml/min. 200 μl of each 1.8 ml fraction was dried in the Speedvac, resuspended in 10 μl water and tested for activity against E. coli, M. luteus and F. graminearum. The fraction of interest was diluted in an equal volume of 0.05% TFA and loaded onto a Macrosphere C8, 5 μm, 300 Å, 250×4.6 mm analytical column (Alltech) equilibrated in 15% solvent B on the HPLC. The column was eluted with a gradient of 15-55% solvent B running over 60 min at 1 ml/min. 200 μl of each 1.8 ml fraction was dried in the Speedvac, resuspended in 10 μl water and tested for activity against E. coli, M. luteus and F. graminearum. The fraction of interest was diluted in an equal volume of 0.05% TFA and loaded onto a μRPC C2/C18, 3 μm , 100×2.1 mm analytical column (Amersham Biosciences) equilibrated in solvent A running on a SMART system (Amersham Biosciences) monitoring at 215, 254 and 280 nm. The column was eluted with a gradient of 0-100% solvent B running over 25 min at 200 μl/min. 50 μl of each 200 μl fraction was dried in the Speedvac, resuspended in 3 μl water and tested for activity against F. graminearum.

For Gm-moricinC1, the fraction of interest was diluted in an equal volume of 0.05% TFA and loaded onto a Prosphere C18, 5 μm, 300 Å, 250×4.6 mm analytical column (Alltech) equilibrated in 10% solvent B on the HPLC. The column was eluted with a gradient of 10-50% B running over 60 min at 1 ml/min. 400 μl of each 1.8 ml fraction was dried in the Speedvac, resuspended in 10 μl water and tested for activity against F. graminearum. The fractions of interest were pooled and diluted in an equal volume of 0.05% TFA and loaded onto the C2/C18 column. The column was equilibrated in solvent A running on the SMART system and was eluted with a gradient of 0-100% solvent B running over 25 min at 200 μl/min while monitoring at 215, 254 and 280 nm. Fractions were collected by peak detection and tested directly against F. graminearum.

### Peptide Identification

The fractions of interest were analysed on a Voyager Elite MALDI-TOF mass spectrometer (Perseptive Biosystems) using 0.5 μl of sample plus 0.5 μl of matrix. For linear mode spectra the matrix was sinapinic acid and the standard was a mixture of cecropin A and myoglobin, and for reflector mode spectra the matrix was α-cyano-4-hydroxycinnamic acid and the standard was a tryptic digest of bovine serum albumin. For N-terminal amino acid sequencing the purified peptides were dried onto fibre glass disks and subject to Edman degradation using a Procise Model 492 Protein Sequencer (Applied Biosystems), in accordance with the manufacturers instructions.

### Results and Discussion

Two different batches of crude hemolymph were processed by C18 solid phase extraction and C18 semi-preparative chromatography. The samples obtained after partial purification by C18 solid phase extraction showed activity against E. coli, M. luteus, F. graminearum, A. alternata, A. rabiei, C. gloeosporioides, L. maculans and A. niger. Further purification of samples on a C18 semi-preparative column produced fractions eluting between approximately 25-40% acetonitrile that showed activity against the test organism F. graminearum. Three fractions from different positions in the gradient were purified further on a C18 analytical column. For Gm-moricinA, three fractions were obtained which had activity against F. graminearum. One of these fractions was purified further on a C8 analytical column, producing one fraction which had activity against F. graminearum. It also showed activity against L. maculans and A. rabiei. This fraction appeared sufficiently pure as judged by mass spectroscopy, that it was subjected to Edman sequencing without further purification.

For Gm-moricinB, purification on the C18 analytical column resulted in one fraction that showed activity against F. graminearum, so this fraction was purified further on a C8 analytical column, resulting in one fraction which had activity against F. graminearum. This fraction was further purified on a C2/C18 column, resulting in one fraction which showed activity against F. graminearum. This fraction appeared sufficiently pure by mass spectroscopy and was sequenced by Edman degradation.

For Gm-moricinC1, purification on the C18 analytical column resulted in two fractions that showed activity against F. graminearum. These fractions were pooled and purified further on a C2/C18 column, resulting in three fractions which had activity against F. graminearum. One of these fractions was judged sufficiently pure by mass spectroscopy for sequencing by Edman degradation.

MALDI mass spectroscopy and Edman sequencing were used to identify the purified peptides. Gm-moricinA had an apparent molecular weight of 4242.9 and a partial amino acid sequence of KVNVNAIKKGGKAIGKGFKVISAASTAHDVYE (SEQ ID NO:19). For Gm-moricinB, the mass spectrum consisted of one major peak (3569) plus several minor components, so it was not possible to be certain of the molecular weight of the active component. The amino acid sequence of the main component was determined to be GGQIIGKALRGINIASTAHDIISQFKPK (SEQ ID NO:20). Gm-moricinC1 had an apparent molecular mass of 3924.4 Da and a partial amino acid sequence of KVPIGAIKKGGKIIKKGLGVIGAAGTAHEVYS (SEQ ID NO: 30). Searches of the non-redundant databases using BLASTP for short matches indicated that these three peptides had some homology to the known peptides moricin from Bombyx mori, Manduca sexta and Spodoptera litura, and virescein from Heliothis virescens.

### Example 2

### Identification of cDNAs Encoding G. mellonella Moricin-Like Peptides

### Preparation of Total RNA and Poly(A)⁺ RNA

Fat body tissue was dissected from G. mellonella larvae at 24 hours after injection with E. coli and M. luteus cell suspension. Larvae that had been chilled on ice for at least 30 min were pinned in a Sylgard dish under ice-cold PBS and opened by a longitudinal incision down the dorsal midline. The gut was removed and fat body was collected with fine watch-makers forceps. Dissected fat body was briefly blotted on absorbent tissue and snap-frozen in a microfuge tube held in liquid nitrogen. The frozen tissues were stored at −80° C.

Total RNA was isolated using Trizol reagent (Astral Scientific). Briefly, approximately 500 mg of frozen fat body tissue was resuspended in 1 mL of Trizol reagent and homogenised in a Polytron tissue homogeniser.

Polyadenylated RNA was isolated by two rounds of selection on oligo(dT)-cellulose spun-column chromatography using the mRNA purification kit (Amersham Biosciences). Approximately 1 mg of total RNA was bound to an oligo(dT)-cellulose spin column, washed and eluted in 1 mL of low salt buffer according to the manufacturer's instructions. The eluted RNA was bound to a second spin column, washed and eluted as described above in a final volume of 1 mL. The mRNA was precipitated by addition of sodium acetate to a final concentration of 0.1M with 200 μL ethanol. The mRNA was recovered by centrifugation and resuspended in 5 μL of DEPC-treated water.

### Preparation of a cDNA Library

A cDNA library was prepared from approximately 5 μg of mRNA using a Lambda UniZap cDNA synthesis and cloning system (Stratagene). Purified cDNA (approx. 20 ng) was ligated to 1 μg of vector DNA and packaged with Gigapack® III Gold packaging extract (Stratagene) to yield a cDNA library with a titre of 5×10⁵ plaque forming units per mL.

### Identification of cDNA Encoding Moricin-like Peptides by PCR

Short, minimally degenerate oligonucleotides were designed by reverse translation of the amino-acid sequences of the G. mellonella Gm-moricinA and Gm-moricinB peptides. The sequences of these oligonucleotides are given in the Table 2.

TABLE 2

Primers used for identification of G. mellonella
moricin cDNAs

Primer

| | | | |
|---|---|---|---|
| Peptide | name | Orientation | Sequence† |

| | | | |
|---|---|---|---|
| Gm-moricinA | GmAF1 | sense | 5′-AAYGTIAAYGCIATHAARAARGG-3′ |
| | | strand | (SEQ ID NO: 21) |
| Gm-moricinA | GmAF2 | antisense | 5′-YTCRTAIACRGCRTGIGCNTG-3′ |
| | | strand | (SEQ ID NO: 22) |
| Gm-moricinB | GmAF3 | sense | 5′-GGIGGICARATHATHGGIAARGC-3′ |
| | | strand | (SEQ ID NO: 23) |
| Gm-moricinB | GmAF4 | antisense | 5′-TGISIDATDATRTCRTGIGCNGT-3′ |
| | | strand | (SEQ ID NO: 24) |

†deoxy-inosine triphosphate (I) was used in some positions to reduce overall degeneracy of the oligonucleotide pool.

Each of the oligonucleotide pairs GmAF1/GmAF2 and GmAF3/GmAF4 were used as primers for PCR amplification of fragments of cDNA encoding the Gm-moricinA and Gm-moricinB peptides. Approximately 2 ng of the purified double-stranded cDNA used for library construction was used as template for the PCR reactions.

PCR products of the expected size were excised from acrylamide gels and the DNA was eluted in ammonium acetate buffer and recovered by ethanol precipitation. The purified DNA was ligated into the cloning vector pGEM-Teasy (Promega) and transformed by electroporation into E. coli DH10B cells. Resultant bacterial colonies were screened for inserts by PCR using primers based on the T7 and SP6 promoter sequences flanking the multiple cloning site of the vector. For each of the Gm-moricinA and Gm-moricinB PCR products, several clones containing inserts of the expected size were sequenced on both strands and the deduced protein sequence was used to verify the clones as true Gm-moricinA and Gm-moricinB cDNA products. A representative clone was selected for each moricin cDNA type for subsequent use in library screening.

### Probe Synthesis

Probes were synthesised in PCR reactions in which the dATP was replaced by radio-labelled dATP. Unique sequence primers were designed from a representative clone for each of the Gm-moricinA and Gm-moricinB cDNA fragments (Table 3). Hybridisation probes were prepared for each of the G. mellonella moricin cDNAs using the primers described in Table 3 and the cDNA inserts of clones pGmmA7 (Gm-moricinA) and pGmmB11 (Gm-moricinB) as template.

TABLE 3

Partial clones and oligonucleotide primers used
for preparation of hybridisation probes for
library screening

Primer

| | | | |
|---|---|---|---|
| Clone | name | Orientation | Sequence |

| | | | |
|---|---|---|---|
| pGmmA7 | GmLM1 | sense | 5′-GAGGAAAGGCCATAGGAAAAGG-3′ |
| | | strand | (SEQ ID NO: 25) |
| pGmmA7 | GmLM2 | antisense | 5′-ACTCGCCGCACTGATTAC-3′ |
| | | strand | (SEQ ID NO: 26) |
| pGmmB11 | GmSM1 | sense | 5′-GGGGGGCAGATCATTGGG-3′ |
| | | strand | (SEQ ID NO: 27) |
| pGmmB11 | GmSM2 | antisense | 5′-TTATGTCATGGGCCGTACT-3′ |
| | | strand | (SEQ ID NO: 28) |

The compositions of the PCR reactions for each of the two probes are described in Table 4. Reaction conditions consisted of an initial denaturing step of 94° C. for 3 min, followed by 30 cycles of 94° C. for 45 sec, 53° C. for 30 sec, 72° C. for 45 sec, and a final step of 72° C. for 5 min to end.

TABLE 4
Probe labelling reaction conditions

| | Gm-moricinA | Gm-moricinB |
|---|---|---|
| Reagent | Probe | Probe |
| 10x PCR buffer | 5 μl | 5 μl |
| 50 mM MgCl₂ | 1.5 μl | 1.5 μl |
| 10 mm dNTPs (mix of dCTP, | 1 μl | 1 μl |
| dGTP, dTTP) | | |
| 10 μM sense strand primer | 3 μl GmLM1 | 6 μl GmSM1 |
| 10 μM anti-sense strand primer | 3 μl GmLM2 | 6 μl GmSM2 |
| ddH₂O | 35 μl | 30 μl |
| Template | 1 μl pGmmA7 | 1 μl pGmmB11 |
| | insert | insert |
| | (1 in 10 dilution) | (1 in 10 dilution) |
| α-(³²P)-dATP | 5 μl | 5 μl |
| Taq Polymerase (5 U/μl) | 0.5 μl | 0.5 μl |

### Library Screening

The cDNA library was plated onto five 15 cm LB agar plates at a density of 5×10⁴ pfu per plate. Duplicate plaque lifts were made on nitrocellulose filters, and the DNA was denatured and fixed to the membranes using standard procedures (Sambrook et al., 1989, supra).

The library was screened first with the probe for the G. mellonella Gm-moricinA gene and the primary filters were washed and re-screened with the probe for the G. mellonella Gm-moricinB gene.

Filters were placed in hybridisation bottles (Hybaid) and prehybridised for a minimum of 2 hours at 60° C. in 20 ml of a solution containing 5×SSPE, 5× Denhardt's solution, 0.5% w/v SDS, and 200 μg/ml freshly denatured herring sperm DNA. The probe was denatured by boiling for 10 min followed by chilling on ice. The cooled probe solution was added to the filters and allowed to hybridise overnight at 60° C.

Filters were washed three times in 0.5×SSPE, 0.1% SDS at 60° C. for 30 minutes per change.

### Isolation and Characterisation of cDNA Encoding the G. mellonella Gm-moricinA Peptide

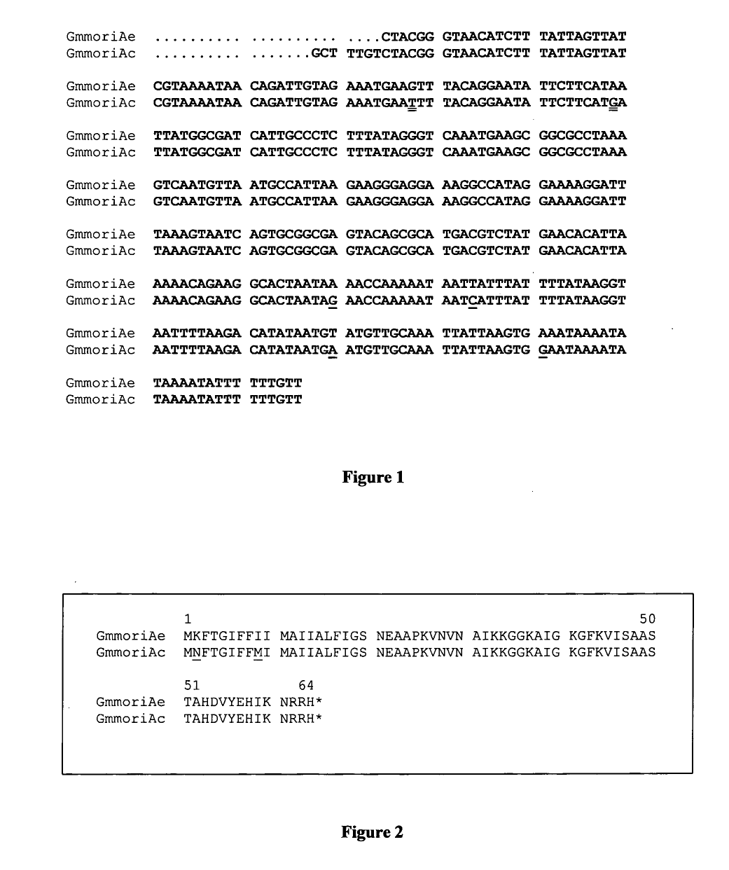

Approximately 80 hybridising phages were identified from the primary library screen. Four of these were plaque-purified, the plasmids excised, and the cDNA inserts sequenced on both strands by dye-terminator sequencing using a Beckman CEQ8000 DNA Analyser and Beckman DCTS chemistry. Three clones were identified (pGmmoriAa, pGmmoriAd, pGmmoriAe) that differed only in length at the 5′-end. The fourth clone (pGmmoriAc) was an allelic variant of the same gene that differed from the other three by 6 nucleotide substitutions, two of which resulted in amino acid replacements in the presumptive secretion signal peptide. The other changes were either silent or occurred in untranslated regions of the mRNA. The nucleotide sequences of two of these Gm-moricinA cDNA clones are shown as a sequence alignment in FIG. 1, and the deduced amino acid sequences are presented as a sequence alignment in FIG. 2.

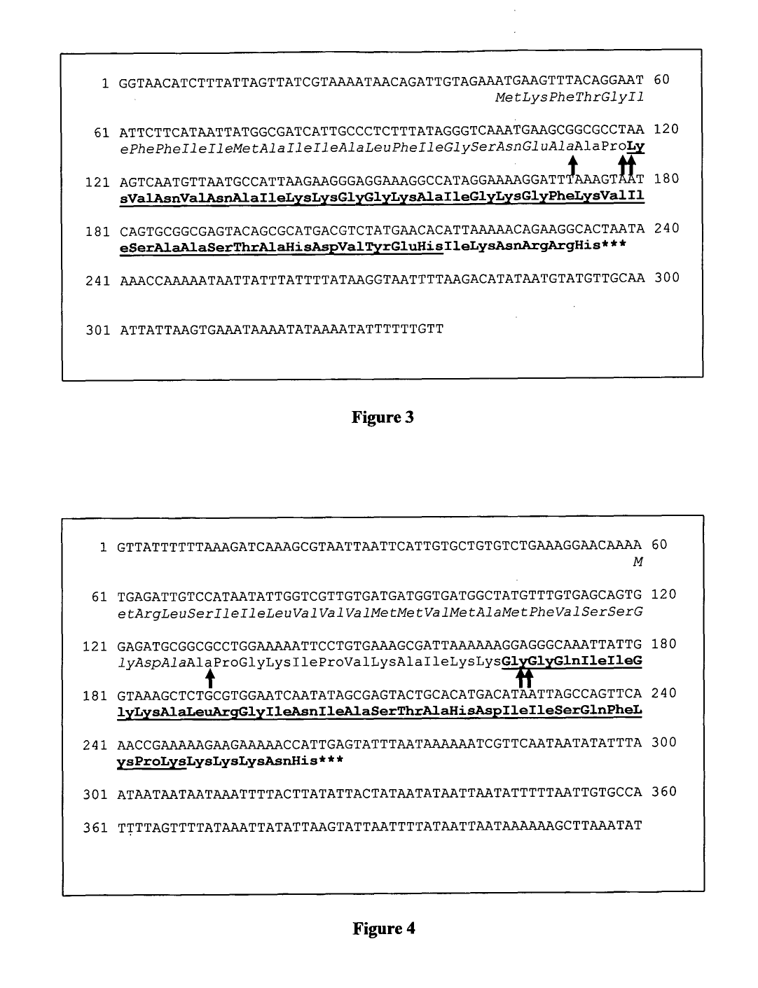

The nucleotide sequence of clone pGmmoriAa, representing the most common clone class, is shown in FIG. 3, together with the deduced protein sequence of the open reading frame encoding the Gm-moricinA peptide, commencing at the first in-frame methionine residue. Sites of predicted protein precursor processing are also indicated on the figure.

### Isolation and Characterisation of cDNA Encoding the G. mellonella Gm-moricinB Peptide

In excess of 150 hybridising phages were identified from the primary library screen. Three clones were plaque-purified, the plasmids excised, and the cDNA inserts sequenced on both strands by dye-terminator sequencing using a Beckman CEQ8000 DNA Analyser and Beckman DCTS chemistry. These clones, designated pGmmoriBe1, pGmmoriBe2 and pGmmoriBd1, differed only in length at the 5′-end.

The nucleotide sequence of a representative clone, pGmmoriBe1, is shown in FIG. 4, together with the deduced protein sequence of the open reading frame encoding the G. mellonella Gm-moricinB peptide, commencing at the first in-frame methionine residue.

### Identification of Gm-moricinC1 and Gm-moricinC2 by PCR from the cDNA Library

The amino acid sequence of Gm-moricinC1 obtained by peptide sequencing was used to design degenerate primers (Gm3-1 and Gm3-2, see Table 5) in order to isolate the gene by PCR from the G. mellonella cDNA library. Sequencing of PCR products identified two forms of the Gm-moricinC gene. Specific primers (GmC1-F, GmC1-R, GmC2-F and GmC2-R, see Table 5) were designed which when used in nested PCR with vector primers based on pBluescript SK phagemid (Stratagene) would distinguish between the two forms of the gene.

For Gm-moricinC1, a 3′ gene fragment was obtained by nested PCR using the primer pairs Gm3-1/M13 reverse and GmC1-F/T3, and a 5′ fragment was obtained by nested PCR using the primer pairs Gm3-2/M13 forward and GmC1-R/T7. For Gm-moricinC2, a 3′ gene fragment was obtained by nested PCR using the primer pairs Gm3-1/M13 reverse and GmC2-F/T3, and a 5′ fragment was obtained by nested PCR using the primer pairs Gm3-2/M13 forward and GmC2-R/T7. The resultant PCR products were sequenced, and included sequence from the 5′ and 3′ untranslated region of the genes.

A third set of specific primers (GmClutr5, GmClutr3, GmC2utr5 and GmC2utr3, see Table 5) were then designed to anneal to the 5′ and 3′ untranslated regions of the two genes. PCR with the primer pairs GmClutr5/GmClutr3 and GmC2utr5/GmC2utr3 allowed the determination of the full sequence of the Gm-moricinC1 and Gm-moricinC2 peptide open reading frames.

For Gm-moricinC1, eight clones were sequenced which apart from variations in length differed at only three nucleotide positions. Two of these substitutions were within the peptide open reading frame, one of which resulted in an amino acid change in the presumptive secretion signal peptide (residue 13, MET or VAL). The nucleotide sequence of a representative clone of Gm-moricinC1, Gm3-01ae, is shown in FIG. 5, together with the deduced protein sequence of the open reading frame commencing at the first in-frame methionine residue. Sites of predicted protein precursor processing are indicated on the figure. The figure also shows the three sites in the sequence where nucleotide substitutions were found, and indicates the amino acid variation found at position 13 in the peptide open reading frame.

TABLE 5

Primers used for identification of the G. mellonella
Gm-moricinC1 and Gm-moricinC2 genes

Primer

| | | | |
|---|---|---|---|
| Peptide | name | Orientation | Sequence |

| | | | |
|---|---|---|---|
| Gm-moricinC | Gm3-1 | sense | 5′-CCNAARGTICCIATHGGNGC-3′ |
| | | strand | (SEQ ID NO: 31) |
| Gm-moricinC | Gm3-2 | antisense | 5′-TANACTTCRTGIGCDGTNCC-3′ |
| | | strand | (SEQ ID NO: 32) |
| Gm-moricinC1 | GmC1-F | sense | 5′-AGGTCTTGGTGTAATTGGTG-3′ |
| | | strand | (SEQ ID NO: 33) |
| Gm-moricinC1 | GmC1-R | antisense | 5′-GCAGCACCAATTACACCAAG-3′ |
| | | strand | (SEQ ID NO: 34) |
| Gm-moricinC2 | GmC2-F | sense | 5′-TAAAAAGGGTCTAGGTGTGC-3′ |
| | | strand | (SEQ ID NO: 35) |
| Gm-moricinC2 | GmC2-R | antisense | 5′-GCGGCGCCAAGCACACCTAG-3′ |
| | | strand | (SEQ ID NO: 36) |
| Gm-moricinC1 | GmC1utr5 | sense | 5′-CTTCAATCTTAGTGAAAACTTCGC-3′ |
| | | strand | (SEQ ID NO: 37) |
| Gm-moricinC1 | GmC1utr3 | antisense | 5′-GGATAGTACTTCATAATTATATAC-3′ |
| | | strand | (SEQ ID NO: 38) |
| Gm-moricinC2 | GmC2utr5 | sense | 5′-GTTGCAGGACTTAATACTTAGTG-3′ |
| | | strand | (SEQ ID NO: 39) |
| Gm-moricinC2 | GmC2utr3 | antisense | 5′-GAGTATTTTACTAATAAGTATGTGG-3′ |
| | | strand | (SEQ ID NO: 40) |

For Gm-moricinC2, no nucleotide variation was observed in four independent clones. The nucleotide sequence of a representative clone of Gm-moricinC2, Gm3-03, is shown in FIG. 6, together with the deduced protein sequence of the open reading frame commencing at the first in-frame methionine residue. Sites of predicted protein precursor processing are also indicated on the figure.

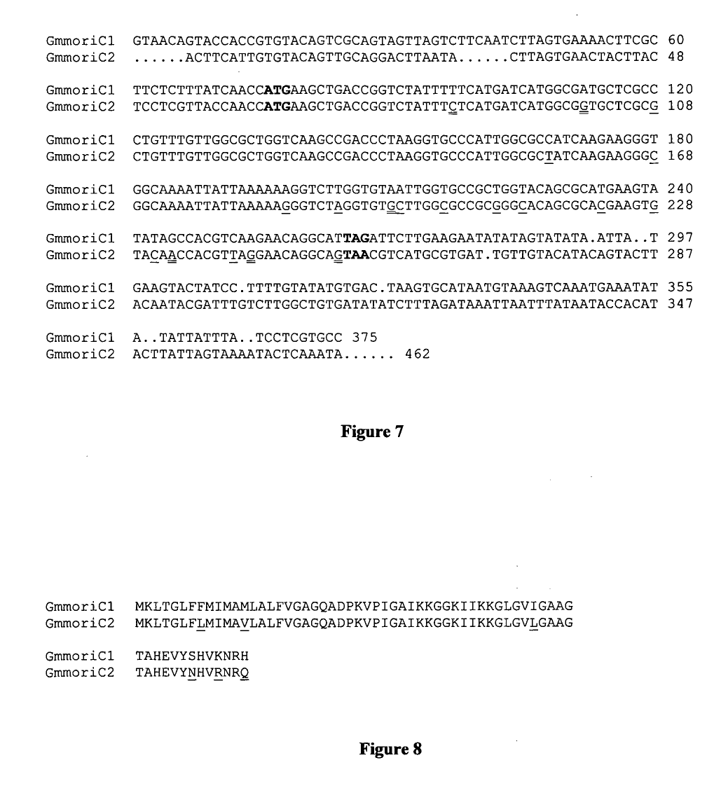

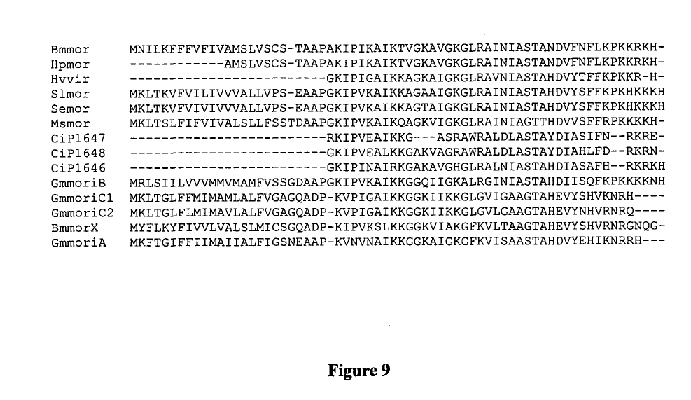

An alignment of Gm-moricinC1 and Gm-moricinC2 at the nucleotide and amino acid level is shown in FIGS. 7 and 8, respectively. At the nucleotide level, Gm-moricinC1 and Gm-moricinC2 differ by 19 nucleotides in the open reading frame, with 3 in the presumptive signal region, and 2 and 14 in the N- and C-terminal halves of the mature peptide, respectively (FIG. 8). At the amino acid level, Gm-moricinC1 and Gm-moricinC2 differ by up to 6 amino acids, including 1-2 in the presumptive signal region (depending on the form of Gm-moricinC1), and 4 in the peptide coding region (FIG. 9). The amino acid differences in the coding region are all in the C-terminal half of the peptide, which is the region where the nucleotide sequences diverge significantly.

### Structural Analysis

Analysis of the B. mori moricin NMR structure (Hemmi et al., 2002) was performed using DSModeling 1.1 (Accelrys Inc). Homology models of Gm-moricinA and B. mori moricinX were built using B. mori moricin as the template and the default settings in DSModeling. Secondary structure predictions were performed using PSIPRED (McGuffin et al., 2000). Phylogenetic trees were constructed using ClustalW (Chenna, et al., 2003) with enbocin as an outlier.

### Discussion

For Gm-moricinA, Gm-moricinB and Gm-moricinC1, the partial sequences determined by N-terminal amino acid sequencing were identical to sections of the translated DNA sequences. This allowed extraction of the complete sequences of the peptides from the predicted open reading frames of the corresponding DNA sequences. Gm-moricinC2, a peptide closely related to Gm-moricinC1, was also identified by PCR on the cDNA library. The deduced sequences of the mature peptides were used to search the non-redundant databases using BLASTP for short matches. These searches indicated that these peptides had similarity to the moricin and virescein peptides previously identified in other Lepidoptera including Bombyx mori, Manduca sexta, Spodoptera litura and Heliothis virescens. A GAP alignment of the G. mellonella peptides with these four peptides indicated that the identity was 77% for the active peptide. Generally, the highest similarity was shown to virescein from H. virescens, and Gm-moricinB was more similar to the known moricins than Gm-moricinA, Gm-moricinC1 or Gm-moricinC2.

The ClustalW alignment of Gm-moricinA, Gm-moricinB, Gm-moricinC1 and Gm-moricinC2 with related peptides from other Lepidoptera is shown in FIG. 9. By combining information from this alignment, the amino acid sequencing, and knowledge about signal peptide processing of insect antimicrobial peptides (Boman et al., 1989), the mature peptides in G. mellonella are most likely to start at residue 26 (FIG. 9). The exception to this is Gm-moricinB, where the N-terminal amino acid sequence and mass spectroscopy data indicate that the active peptide isolated from G. mellonella hemolymph starts at residue 36.

The main feature of the B. mori moricin structure, as discussed in the literature (Hemmi et al., 2002) and further analysed using DSModeling, is a helix from residues 5-36 with a bend between residues 22 and 23. It is interesting to correlate this structural information with that of the peptide sequence alignment shown in FIG. 9. The helix starts at position 5 (relative to the N-terminus of the mature protein), immediately after a proline residue that is present in every moricin sequence except for Gm-moricinA. For Gm-moricinA, secondary structure predictions using PSIPRED suggested that a helix starts at the equivalent position (residue 4) as for the other moricins, despite the lack of a proline residue. The N-terminal region (1-22) of the B. mori moricin is amphipathic, with 6 basic amino acids on one face of the helix. The other moricins contain 5-7 basic residues in the same region, although the sequence position of the basic amino acids is not conserved. This suggests that it is the positively charged helical face which is the important feature rather than the exact sequence position of the basic residues. Gm-moricinB is an interesting case, as the active peptide has a significantly less positively charged N-terminal region due to the truncation of ten residues. For the B. mori moricin, the C-terminal helical region (23-36) is hydrophobic, with a totally conserved acidic residue in the middle of the helical region. Gm-moricinA and the C. illioneus moricin P1648 are interesting in that they contain an extra acidic residue at the end of this C-terminal helical region, and the Gm-moricinA model shows that the two acidic residues cluster on one face of the helix. The B. mori moricin and about half of the known moricin sequences have a proline residue at the end of the helical region. The Gm-moricinA helix is also predicted to finish at the same point despite the lack of the proline residue. For the B. mori moricin, the C-terminal tail is unstructured. In all moricins this tail is highly charged and is a feature which distinguishes the moricins from the cecropins (Hemmi et al).

### Example 3

### Activity of Synthetic G. mellonella Gm-moricinA, Gm-moricinB and Gm-moricinC2 Against Various Fungi

Four moricin peptides were synthesised by Auspep (Melbourne, Australia) using standard peptide synthesis techniques. These were B. mori moricin (residues 25 to 66 of SEQ ID NO:16), Gm-moricinA (SEQ ID NO:4), Gm-moricinB (SEQ ID NO:5) and Gm-moricinC2 (SEQ ID NO:53). The peptides were tested for activity against the bacteria E. coli and M. luteus, and against spores of the fungi F. graminearum, F. oxysporum, A. rabiei, C. gloeosporioides, L. maculans and A. niger generally as described in Example 1. The concentrations tested were 0.1, 1, 10 and 100 μM, and 1 μg/μl. Results are shown in Table 6, and indicate that all peptides showed some antifungal activity.

The synthetic peptides were also tested against fungal mycelia using the inhibition zone plate assay. Mycelia from F. graminearum and F. oxysporum were fragmented by grinding with a mini-pestle in sterile water in a microfuge tube. Fungal plates were prepared using YPD broth containing 0.8% agarose and a volume of mycelial fragments that resulted in uniform growth of the fungus. The sample of interest (2 μl) was spotted on the surface of the plate, and the organism grown under appropriate conditions. Results are summarised in Table 6, and indicate that the moricin peptides are also active against fungal mycelia.

TABLE 6
Activity of synthetic moricin peptides against various microorganisms.
The indicated concentration (μM) is the lowest concentration at which
activity was observed in the zone inhibition assay, an N indicates that no
activity was observed, and a dash indicates that the sample was not tested.

| peptide | Eco | Mlu | Fgr | Fgm | Fox | Fom | Ara | Lma | Ani | Cgl |
|---|---|---|---|---|---|---|---|---|---|---|
| Bm-moricin | 10 | 10 | 10 | 180 | 100 | 100 | 100 | 100 | N | N |
| Gm-moricinA | 10 | 10 | 1 | 100 | 10 | 10 | 1 | 10 | N | N |
| Gm-moricinB | 100 | 100 | 10 | 280 | 100 | 280 | — | 100 | N | N |
| Gm-moricinC2 | 10 | 100 | 1 | 10 | — | 10 | — | 10 | N | — |

Eco, Escherichia coli;
Mlu, Micrococcus luteus;
Fgr, Fusarium graminearum spores;
Fgm, F. graminearum mycelia;
Fox, Fusarium oxysporum spores;
Fom, F. oxysporummycelia;
Ara, Ascochyta rabiei spores;
Cgl, Colletotrichum gloeosporioides spores;
Lma, Leptosphaeria maculans spores;
Ani, Aspergillus niger spores.

Synthetic Gm-moricinA was also tested against six yeast species using the NCCLS M27-A2 microbroth dilution method at the Women's and Children's Hospital (Adelaide, Australia). The yeasts tested were Candida albicans, C. parapsilosis, C. glabrata, C. krusei, C. tropicalis and Cryptococcus neoformans. The peptide was tested at 0.125-64 μg/ml, with each yeast species tested in duplicate. Trays were read at 24, 48 or 72 hours as appropriate. The MIC₉₀ values determined were 4.0 μg/ml for C. parapsilosis, C. tropicalis and C. neoformans, 8.0 μg/ml for C. krusei, and 64.0 μg/ml for C. albicans and C. glabrata. These results indicate that Gm-moricinA has activity against yeasts.

Construction of a phylogenetic tree based on the ClustalW alignment of the mature peptide sequences (FIG. 9) indicated that GmmoricinA, GmmoricinC1, GmmoricinC2 and BmmoricinX were closely related and could be considered to cluster together as a sub-family of the moricins. Antifungal testing of two members of this sub-family (synthetic Gm-moricinA and Gm-moricinC2) suggested that this group of peptides had better antifungal activity than the synthetic B. mori moricin (Table 6).

### Example 4

### Expression of Antifungal Peptides in Arabidopsis

### Agrobacterium-mediated Transformation of Arabidopsis with the G. mellonella Gm-moricinA Gene

DNA encoding Gm-moricinA was cloned into the Agrobacterium transfer vector, p277 (obtained from CSIRO Plant Industry, Canberra, Australia). This vector was constructed by inserting the NotI frag from pART7 into pART27 (Gleave, 1992). The p277 vector contains the CaMV 35S promoter and OCS terminator for plant expression, markers for antibiotic selection, and the sequences required for plant transformation. Three Gm-moricinA DNA constructs were chosen for transformation into Arabidopsis thaliana—the mature Gm-moricinA with no signal peptide, the full-length Gm-moricinA including its native signal peptide, and a fusion consisting of an Arabidopsis vacuolar basic chitinase signal peptide and the mature Gm-moricinA sequence. These constructs were synthesised by PCR and directionally cloned into the p277 transfer plasmid.

Transformation of the Agrobacterium strain GV3101 was achieved using the triparental mating method. This involved co-streaking cultures of A. tumefasciens GV3101, E. coli carrying a helper plasmid, RK2013, and E. coli carrying the desired recombinant p277 plasmid onto a non-selective LB plate. Overnight incubation at 28° C. resulted in a mixed culture which was collected and dilution streaked onto LB plates which selected for A. tumefasciens GV3101 carrying the p277 recombinant plasmid.

Arabidopsis plants were cultured by standard methods at 23° C. with an 18 hr light period per day. Transformation of Arabidopsis plants was carried out by floral dipping. Plants were grown to an age, 3-5 weeks, where there were many flower stems presenting flowers at various stages of development. An overnight culture of transformed A. tumefasciens GV3101 was pelleted and resuspended in 5% sucrose containing the wetting agent Silwet-77. Flowers were dipped into the bacterial suspension and thoroughly wetted by using a sweeping motion. The plants were wrapped in plastic film and left overnight on a bench top at room temperature, before being unwrapped and placed back into a plant growth cabinet maintained at 21° C. The dipping was repeated 1-2 weeks later to increase the number of transformed seeds. The seeds were collected 3-4 weeks after dipping, dried in seed envelopes for the appropriate length of time for each ecotype, then sterilised and germinated on Noble agar plates containing selective antibiotics and an antifungal agent.

Positive transformants were transplanted into Arasystem pots (Betatech), grown to maturity inside Aracon system sleeves and the seeds carefully collected. Thirty two transformed Arabidopsis plants (T1 generation) were screened by PCR and reverse-transcriptase PCR (RT-PCR) to confirm the presence and expression of the recombinant gene. Genomic DNA was extracted from the leaves of plants transformed with the full-length Gm-moricinA construct using the Extract-N-Amp Plant PCR and Extract-N-Amp Reagent kits (Sigma). PCR on the extracts was performed using primers specific to the Gm-moricinA gene (LMxho5, 5′-CTCGAGAACAATGAAGTTTACAGGAATATTCTTCA-3′ (SEQ ID NO: 41) and LMxba3, 5′-TCTAGATTAGTGCCTTCTGTTTTTAATGTGTTCATAGAC-3′ (SEQ ID NO: 42)). For RT-PCR, 8 plants transformed with the full-length Gm-moricinA construct were randomly selected for analysis. Leaves from these plants were snap frozen and ground in liquid nitrogen using a mortar and pestle. RNA was isolated using the RNeasy Plant kit (Qiagen). cDNA was prepared from the RNA using the iScript cDNA Synthesis kit (Bio-Rad). PCR was performed using 1 μl of cDNA, recombinant Taq polymerase (Invitrogen), an annealing temperature of 54° C., and Gm-moricinA specific primers (LMxho5 and LMxba3). 3 μl of each 25 μl PCR reaction was visualised on a 1.2% agarose gel.

T1 seedlings can be transplanted and cultivated for seed through two generations to eventually isolate the homozygous T3 seeds. T3 plants can then be screened for increased resistance to fungal disease.

### Inoculation Protocol using Fusarium oxysporum

A Fusarium oxysporum strain known to be pathogenic to Arabidopsis was obtained from J. Manners (CSIRO Plant Industry, Queensland, Australia). The fungal isolate was maintained on ½ strength Potato Dextrose Agar (PDA).

From maintenance stocks, cores were taken and used to inoculate 500 ml Potato Dextrose Broth (PDB). Flasks were incubated on a shaker for 7 days at 28° C. The inoculum was drained through miracloth prior to quantification with a haemocytometer. The spores were diluted with sterile distilled water and used to inoculate Arabidopsis strains.

Several ecotypes of Arabidopsis were cultivated for testing, including Columbia 0 (Col-0), Landsberg erecta (L-er) and Sg-1 (obtained from CSIRO Plant Industry, Canberra, Australia). Arabidopsis plants used in the inoculation were grown singly in ‘jiffy’ pots for approximately 2-3 weeks. Watering of plants was ceased approximately 4 days prior to infection. Arabidopsis plants were inoculated by adding the spores (5 ml of 2×10⁶-1×10⁷ spores/ml) directly onto the soil near the plant stem. Plants were incubated at 25° C. and scored for wilt symptoms and/or death approximately 10-12 days post inoculation.

To further characterize the level of disease caused to a specific genotype, a set of oligonucleotide primers (Fo18Sits-F, 5′-CGCCAGAGGACCCCTAAAC-3′ (SEQ ID NO: 43) and Fo18Sits-R, 5′-ATCGATGCCAGAACCAAGAGA-3′ (SEQ ID NO:44)) were used to amplify a region of 18S rRNA from F. oxysporum. The primers demonstrate little to no homology with Arabidopsis RNA and act to indicate the difference in fungal RNA levels as compared to the amount of plant RNA.

### Results and Discussion

All three Arabidopsis ecotypes (Col-0, L-er, Sg-1) showed disease symptoms when infected with F. oxysporum. The most obvious disease phenotype was observed with the Sg-1 ecotype with a response evident by 6 days post infection. Quantitative 15 PCR on RNA extracted from the leaf material of infected Col-0 and L-er plants showed the presence of F. oxysporum RNA even when the disease phenotype of the plants was poor, confirming that infection had occurred.

Arabidopsis plants (Sg-1 ecotpye) were transformed with the three Gm-moricinA constructs and the seeds germinated under kanamycin selection. The transformation rates (the percentage of T1 seedlings produced as a proportion of the seeds sown) were 0.53, 0.85 and 0.25% for the mature Gm-moricinA with no signal peptide, the full-length Gm-moricinA including its native signal peptide, and the chitinase signal peptide-Gm-moricinA fusion, respectively. PCR on genomic DNA extracted from the leaves of plants transformed with the full-length Gm-moricinA construct indicated that all plants contained the transgene. Further analysis of 8 randomly selected transformants containing the full-length Gm-moricinA gene was conducted by RT-PCR. Transcripts were detected in all 8 lines demonstrating that the Gm-moricinA gene was being efficiently expressed.

Similar procedures as those described herein can readily be used to express other proteins of the invention, such as Gm-moricinB, Gm-moricinC1, and/or Gm-moricinC2 in plants.

### Example 5

### Preparation and Use of G. mellonella Gm-moricinA Antiserum

Antibodies were raised against synthetic Gm-moricinA, using standard procedures (see, for example, Ed Harlow and David Lane (editors) Antibodies: A Laboratory Manual, Cold Spring Harbour Laboratory, (1988)) for subcutaneous injection of New Zealand white rabbits at the Institute for Medical and Veterinary Science (Adelaide, Australia). One rabbit was treated in the standard manner, with a primary inoculation of 1 mg of peptide followed by three 0.83 mg boosts. A second rabbit was inoculated with 0.1 mg of peptide plus aluminium hydroxide, followed by three boosts of 0.83 mg of peptide with aluminium hydroxide. Approximately 40 ml of serum was collected from each rabbit following the final boost. The antiserum was used without further purification, and evaluated by ELISA, dot blot and Western blot.

Protein electrophoresis was performed using 10% Bis-Tris NuPAGE Novex pre-cast gels (Invitrogen) and a MES running buffer, as recommended by the manufacturer. Western blot transfers were performed using a 0.2 μm Trans-Blot nitrocellulose membrane (BioRad) on a Novablot semi-dry blotter at 0.8 mA/cm² in transfer buffer (25 mM Bicine, 25 mM Bis-Tris, 1 mM EDTA, pH 7.2) containing 20% methanol. The membrane was processed at room temperature in TBS containing 0.1% Tween-20 using three 5 min washes between all steps. The steps used were an overnight block with 3% BSA, a 1 hour incubation with the peptide antiserum (1/250-1/500 dilution) and a 1 hour incubation with anti-rabbit IgG alkaline phosphatase conjugate (Sigma, 1/30000 dilution). The blots were visualised using nitroblue tetrazolium (NBT) and 5-bromo-4-chloro-3-indolyl phosphate (BCIP) (Promega) in substrate buffer (100 mM Tris-Cl, 5 mM MgCl₂, 100 mM NaCl, pH 9.5).

In a Western blot it was possible to detect 50 ng, possibly less, of Gm-moricinA on an SDS-PAGE gel at an antiserum dilution of 1/250. The antiserum seemed highly specific, as a Western blot of an SDS-PAGE gel with 1 μg loadings of synthetic peptides only detected Gm-moricinA, and not Gm-moricinB, B. mori moricin, or an unrelated control peptide.

### Example 6

### Expression of G. mellonella Gm-moricinA Peptide in Insect Cells

The Gm-moricinA peptide was expressed in Sf21 cells using a recombinant baculovirus constructed using GATEWAY technology (Invitrogen). Primers were designed containing the attB1 and attB2 recognition sequences linked to eukaryotic control regions and Gm-moricinA-specific sequences (Lm1attB1, 5′-attB1-TCGAAGGAGATGCCACCATGAAGTTTACAGGAATATTCTTCA-3′ (SEQ ID NO:45) and Lm2attB2, 5′-attB2-TTAGTGCCTTCTGTTTTTAATGTGTTCATAGAC-3′ (SEQ ID NO: 46)). These primers were used with Pfx polymerase (Invitrogen) to amplify a Gm-moricinA-attB PCR product. The PCR product (100 fmol) was shuttled via the pDONR201 entry vector (100 fmol) into the pDEST-8 baculovirus destination vector according to the manufacturer's instructions. Transformants were selected on ampicillin plates and plasmids prepared using the FastPlasmid mini plasmid purification kit (Eppendorf). Positive transformants were confirmed by PCR, restriction enzyme digestion and sequence analysis.

The pDEST-8-Gm-moricinA plasmid DNA was transformed into the DH10Bac competent cell line (Invitrogen) by heat shock at 42° C. for 45 s, chilling on ice for 2 min, and growth in SOC medium for 3 hr at 37° C. with shaking. White colonies were selected on LB plates containing tetracycline, gentamycin, kanamycin, isopropyl-β-thiogalactoside (IPTG) and 5-bromo-4-chloro-3-indolyl β-D-galactoside (X-gal) after overnight incubation at 37° C. Bacmid DNA was extracted from cultures grown overnight at 37° C. using the standard ‘Bac-to-Bac’ protocol (Invitrogen), and screened for the Gm-moricinA gene by PCR using M13 forward and reverse primers.

Bacmid DNA was transfected into Sf21 cells using DOTAP liposomal transfection reagent (Roche) and grown in BaculoGold Max-XP serum-free medium (BD Biosciences) in 6 well plates. Incubation at 27° C. for approximately 90 hr resulted in significant cell pathogenesis. The supernatant was removed and clarified, and cellular material was harvested by scraping cells into fresh medium, centrifugation and resuspension in 50 mM Tris pH 6.8.

To analyse Sf21 cell extracts for the presence of Gm-moricinA mRNA, RNA was prepared using the Perfect RNA kit (Eppendorf). RT-PCR was performed using the Superscript II One-Step RT-PCR kit (Gibco) and the Lm2attB1 and Lm2attB2 primers. Cell and supernatant samples were also processed by C18 solid phase extraction and tested for activity against F. graminearum (see Example 1) and for the presence of Gm-moricinA by Western blot using the Gm-moricinA antiserum (see Example 5).

### Results and Discussion

RNA preparations and RT-PCR confirmed the presence of the Gm-moricinA mRNA in Sf21 cell extracts. Cell and supernatant samples processed by C18 solid phase extraction showed activity against F. graminearum. The Western blot results suggested that fully processed Gm-moricinA was being produced and secreted into the supernatant. These results confirm that the baculovirus system can produce correctly processed Gm-moricinA peptide with antifungal activity.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Banzet, N. et al. (2002) Plant Sci., 162; 995-1006.
Boman, H. G. et al. (1989) J. Biol. Chem., 264; 5852-5860.
Chenna, R. et al. (2003) Nucl. Acids Res., 31:3497-3500.
DeLucca, A. J., and Walsh, T. J. (1999) Antimicrob. Agents Chemother., 43; 1-11.
Gleave, A. P. (1992) Plant Mol. Biol., 20; 1203-1207.
Hara, S. and Yamakawa, M. (1995) J. Biol. Chem., 270; 29923-29927.
Hara, S. and Yamakawa, M. (1996) Biochem. Biophys. Res. Commun., 220; 664-669.
Harayama, S. (1998) Trends Biotech., 16; 76-82.
Hemmi, H., Ishibashi, J., Hara, S. and Yamakawa, M. (2002) FEBS Letters, 518; 33-38.
McGuffin, L. J. et al. (2000) Bioinformatics, 16; 404-405.
Needleman, S. B. and Wunsch, C. D. (1970) J. Mol. Biol., 48; 443-453.

1. A substantially purified peptide which comprises a sequence selected from the group consisting of:
i) an amino acid sequence as provided in SEQ ID NO:4,
ii) an amino acid sequence which is at least 60% identical to SEQ ID NO:4,
iii) an amino acid sequence as provided in SEQ ID NO:5,
iv) an amino acid sequence which is at least 80% identical to SEQ ID NO:5,
v) an amino acid sequence as provided in SEQ ID NO:48,
vi) an amino acid sequence which is at least 70% identical to SEQ ID NO:48,
vii) an amino acid sequence as provided in SEQ ID NO:53,
viii) an amino acid sequence which is at least 70% identical to SEQ ID NO:53,
ix) a biologically active fragment of any one of i) to viii), and
x) a precursor comprising the amino acid sequence according to any one of i) to ix),
wherein the peptide, or fragment thereof, exhibits antifungal and/or antibacterial activity.

2-4. (canceled)

5. The peptide of claim 1 which is fused to at least one other polypeptide/peptide sequence.

6. An isolated polynucleotide, the polynucleotide comprising a sequence selected from the group consisting of:
i) a sequence of nucleotides provided in SEQ ID NO:9 or SEQ ID NO:10;
ii) a sequence of nucleotides provided in SEQ ID NO:11;
iii) a sequence of nucleotides provided in SEQ ID NO:12;
iv) a sequence of nucleotides provided in SEQ ID NO:13;
v) a sequence of nucleotides provided in SEQ ID NO:50;
vi) a sequence of nucleotides provided in SEQ ID NO:51;
vii) a sequence of nucleotides provided in SEQ ID NO:55;
viii) a sequence of nucleotides provided in SEQ ID NO:56;
ix) a sequence encoding a peptide according to claim 1;
x) a sequence of nucleotides which is at least 66% identical to SEQ ID NO:9, SEQ ID NO:10, or SEQ ID NO:12;
xi) a sequence of nucleotides which is at least 71% identical to SEQ ID NO:11 or SEQ ID NO:13;
xii) a sequence of nucleotides which is at least 62% identical to SEQ ID NO:50, or SEQ ID NO:51;
xiii) a sequence of nucleotides which is at least 62% identical to SEQ ID NO:55, or SEQ ID NO:56; and
xiv) a sequence which hybridizes to any one of (i) to (viii) under high stringency conditions.

7. (canceled)

8. A vector comprising the polynucleotide of claim 6.

9. A host cell comprising the polynucleotide of claim 6.

10. The host cell of claim 9 which is a plant cell.

11. A process for preparing a substantially purified peptide which comprises a sequence selected from the group consisting of:
i) an amino acid sequence as provided in SEQ ID NO:4,
ii) an amino acid sequence which is at least 60% identical to SEQ ID NO:4,
iii) an amino acid sequence as provided in SEQ ID NO:5,
iv) an amino acid sequence which is at least 80% identical to SEQ ID NO:5,
v) an amino acid sequence as provided in SEQ ID NO:48,
vi) an amino acid sequence which is at least 70% identical to SEQ ID NO:48,
vii) an amino acid sequence as provided in SEQ ID NO:53,
viii) an amino acid sequence which is at least 70% identical to SEQ ID NO:53,
ix) a biologically active fragment of any one of i) to viii), and
x) a precursor comprising the amino acid sequence according to any one of i) to ix),
wherein the peptide, or fragment thereof, exhibits antifungal and/or antibacterial activity, the process comprising cultivating a host cell according to claim 9 or claim 10 under conditions which allow expression of the polynucleotide encoding the peptide, and recovering the expressed peptide as a substantially purified peptide.

12. A composition comprising a peptide of claim 1, and one or more acceptable carriers.

13. A composition comprising a polynucleotide according to claim 6, and one or more acceptable carriers.

14. A method for killing, or inhibiting the growth and/or reproduction of a fungus and/or a bacteria, the method comprising exposing the fungus and/or bacteria to a peptide of claim 1.

15. A transgenic plant, the plant having been transformed with a polynucleotide according to claim 6, wherein the plant produces a peptide which comprises a sequence selected from the group consisting of:
i) an amino acid sequence as provided in SEQ ID NO:4,
ii) an amino acid sequence which is at least 60% identical to SEQ ID NO:4,
iii) an amino acid sequence as provided in SEQ ID NO:5,
iv) an amino acid sequence which is at least 80% identical to SEQ ID NO:5,
v) an amino acid sequence as provided in SEQ ID NO:48,
vi) an amino acid sequence which is at least 70% identical to SEQ ID NO:48,
vii) an amino acid sequence as provided in SEQ ID NO:53,
viii) an amino acid sequence which is at least 70% identical to SEQ ID NO:53,
ix) a biologically active fragment of any one of i) to viii), and
x) a precursor comprising the amino acid sequence according to any one of i) to ix),
wherein the peptide, or fragment thereof, exhibits antifungal and/or antibacterial activity.

16. A method of controlling fungal and/or bacterial infections of a crop, the method comprising cultivating a crop of transgenic plants of claim 15.

17. A transgenic non-human animal, the animal having been transformed with a polynucleotide according to claim 6, wherein the animal produces a peptide which comprises a sequence selected from the group consisting of:
i) an amino acid sequence as provided in SEQ ID NO:4,
ii) an amino acid sequence which is at least 60% identical to SEQ ID NO:4,
iii) an amino acid sequence as provided in SEQ ID NO:5,
iv) an amino acid sequence which is at least 80% identical to SEQ ID NO:5,
v) an amino acid sequence as provided in SEQ ID NO:48,
vi) an amino acid sequence which is at least 70% identical to SEQ ID NO:48,
vii) an amino acid sequence as provided in SEQ ID NO:53,
viii) an amino acid sequence which is at least 70% identical to SEQ ID NO:53,
ix) a biologically active fragment of any one of i) to viii), and
x) a precursor comprising the amino acid sequence according to any one of i) to ix),
wherein the peptide, or fragment thereof, exhibits antifungal and/or antibacterial activity.

18. A method of treating or preventing a fungal and/or bacterial infection in a patient, the method comprising administering to the patient a peptide of claim 1.

19. (canceled)

20. An antibody which specifically binds to a peptide of claim 1.

21. A method for killing, or inhibiting the growth and/or reproduction of a fungus, the method comprising exposing the fungus to a peptide which comprises a sequence selected from the group consisting of:
i) an amino acid sequence comprising residues 25 to 67 of SEQ ID NO:14,
ii) an amino acid sequence as provided in SEQ ID NO:17,
iii) an amino acid sequence comprising residues 26 to 67 of SEQ ID NO:15,
iv) an amino acid sequence which is at least 75% identical to any one of i) to iii),
v) an amino acid sequence comprising residues 26 to 66 of SEQ ID NO:18,
vi) an amino acid sequence which is at least 50% identical to v), and
vii) a biologically active fragment of any one of i) to vi).

22. (canceled)

23. A method of controlling fungal infections of a crop, the method comprising cultivating a crop of transgenic plants which produce a peptide which comprises a sequence selected from the group consisting of:
i) an amino acid sequence comprising residues 25 to 67 of SEQ ID NO:14,
ii) an amino acid sequence comprising residues 25 to 66 of SEQ ID NO:16,
iii) an amino acid sequence as provided in SEQ ID NO:17,
iv) an amino acid sequence comprising residues 26 to 67 of SEQ ID NO:15,
v) an amino acid sequence which is at least 75% identical to any one of i) to iv),
vi) an amino acid sequence comprising residues 26 to 66 of SEQ ID NO:18,
vii) an amino acid sequence which is at least 50% identical to vi), and
viii) a biologically active fragment of any one of i) to vii).

24. (canceled)

25. A method of treating or preventing a fungal infection in a patient, the method comprising administering to the patient a peptide which comprises a sequence selected from the group consisting of:
i) an amino acid sequence comprising residues 25 to 67 of SEQ ID NO:14,
ii) an amino acid sequence as provided in SEQ ID NO:17,
iii) an amino acid sequence comprising residues 26 to 67 of SEQ ID NO:15,
iv) an amino acid sequence which is at least 75% identical to any one of i) to iii),
v) an amino acid sequence comprising residues 26 to 66 of SEQ ID NO:18,
vi) an amino acid sequence which is at least 50% identical to v), and
vii) a biologically active fragment of any one of i) to vi).

26. (canceled)

27. A kit comprising a peptide of claim 1.


SEQUENCE LISTING (62 sequences)

SEQ ID NO: 1 - protein, 64 aa, Galleria mellonella
MKFTGIFFIIMAIIALFIGSNEAAPKVNVNAIKKGGKAIGKGFKVISAASTAHDVYEHIKNRRH

SEQ ID NO: 2 - protein, 64 aa, Galleria mellonella
MNFTGIFFMIMAIIALFIGSNEAAPKVNVNAIKKGGKAIGKGFKVISAASTAHDVYEHIKNRRH

SEQ ID NO: 3 - protein, 68 aa, Galleria mellonella
MRLSIILVVVMMVMAMFVSSGDAAPGKIPVKAIKKGGQIIGKALRGINIASTAHDIISQFKPKKKKNH

SEQ ID NO: 4 - protein, 39 aa, Galleria mellonella
KVNVNAIKKGGKAIGKGFKVISAASTAHDVYEHIKNRRH

SEQ ID NO: 5 - protein, 33 aa, Galleria mellonella
GGQIIGKALRGINIASTAHDIISQFKPKKKKNH

SEQ ID NO: 6 - DNA, 342 bp, Galleria mellonella
ctacgggtaacatctttattagttatcgtaaaataacagattgtagaaatgaagtttacaggaatattcttcataattatggcgatcattgccctctttatagggtcaaatgaagcggcgcctaaagtcaatgttaatgccattaagaagggaggaaaggccataggaaaaggatttaaagtaatcagtgcggcgagtacagcgcatgacgtctatgaacacattaaaaacagaaggcactaataaaaccaaaaataattatttattttataaggtaattttaagacatataatgtatgttgcaaattattaagtgaaataaaatataaaatattttttgtt

SEQ ID NO: 7 - DNA, 349 bp, Galleria mellonella
gctttgtctacgggtaacatctttattagttatcgtaaaataacagattgtagaaatgaattttacaggaatattcttcatgattatggcgatcattgccctctttatagggtcaaatgaagcggcgcctaaagtcaatgttaatgccattaagaagggaggaaaggccataggaaaaggatttaaagtaatcagtgcggcgagtacagcgcatgacgtctatgaacacattaaaaacagaaggcactaatagaaccaaaaataatcatttattttataaggtaattttaagacatataatgaatgttgcaaattattaagtggaataaaatataaaatattttttgtt

SEQ ID NO: 8 - DNA, 420 bp, Galleria mellonella
gttattttttaaagatcaaagcgtaattaattcattgtgctgtgtctgaaaggaacaaaatgagattgtccataatattggtcgttgtgatgatggtgatggctatgtttgtgagcagtggagatgcggcgcctggaaaaattcctgtgaaagcgattaaaaaaggagggcaaattattggtaaagctctgcgtggaatcaatatagcgagtactgcacatgacataattagccagttcaaaccgaaaaagaagaaaaaccattgagtatttaataaaaaatcgttcaataatatatttaataataataataaattttacttatattactataatataattaatatttttaattgtgccattttagttttataaattatattaagtattaattttataattaataaaaaagcttaaatat

SEQ ID NO: 9 - DNA, 192 bp, Galleria mellonella
atgaagtttacaggaatattcttcataattatggcgatcattgccctctttatagggtcaaatgaagcggcgcctaaagtcaatgttaatgccattaagaagggaggaaaggccataggaaaaggatttaaagtaatcagtgcggcgagtacagcgcatgacgtctatgaacacattaaaaacagaaggcac

SEQ ID NO: 10 - DNA, 192 bp, Galleria mellonella
atgaattttacaggaatattcttcatgattatggcgatcattgccctctttatagggtcaaatgaagcggcgcctaaagtcaatgttaatgccattaagaagggaggaaaggccataggaaaaggatttaaagtaatcagtgcggcgagtacagcgcatgacgtctatgaacacattaaaaacagaaggcac

SEQ ID NO: 11 - DNA, 204 bp, Galleria mellonella
atgagattgtccataatattggtcgttgtgatgatggtgatggctatgtttgtgagcagtggagatgcggcgcctggaaaaattcctgtgaaagcgattaaaaaaggagggcaaattattggtaaagctctgcgtggaatcaatatagcgagtactgcacatgacataattagccagttcaaaccgaaaaagaagaaaaaccat

SEQ ID NO: 12 - DNA, 117 bp, Galleria mellonella
aaagtcaatgttaatgccattaagaagggaggaaaggccataggaaaaggatttaaagtaatcagtgcggcgagtacagcgcatgacgtctatgaacacattaaaaacagaaggcac

SEQ ID NO: 13 - DNA, 99 bp, Galleria mellonella
ggagggcaaattattggtaaagctctgcgtggaatcaatatagcgagtactgcacatgacataattagccagttcaaaccgaaaaagaagaaaaaccat

SEQ ID NO: 14 - protein, 67 aa, Spodoptera litura
MKLTKVFVILIVVVALLVPSEAAPGKIPVKAIKKAGAAIGKGLRAINIASTAHDVYSFFKPKHKKKH

SEQ ID NO: 15 - protein, 67 aa, Manduca sexta
MKLTSLFIFVIVALSLLFSSTDAAPGKIPVKAIKQAGKVIGKGLRAINIAGTTHDVVSFFRPKKKKH

SEQ ID NO: 16 - protein, 66 aa, Bombyx mori
MNILKFFFVFIVAMSLVSCSTAAPAKIPIKAIKTVGKAVGKGLRAINIASTANDVFNFLKPKKRKH

SEQ ID NO: 17 - protein, 41 aa, Heliothis virescens
GKIPIGAIKKAGKAIGKGLRAVNIASTAHDVYTFFKPKKRH

SEQ ID NO: 18 - protein, 66 aa, Bombyx mori
MYFLKYFIVVLVALSLMICSGQADPKIPVKSLKKGGKVIAKGFKVLTAAGTAHEVYSHVRNRGNQG

SEQ ID NO: 19 - protein, 32 aa, Galleria mellonella
KVNVNAIKKGGKAIGKGFKVISAASTAHDVYE

SEQ ID NO: 20 - protein, 28 aa, Galleria mellonella
GGQIIGKALRGINIASTAHDIISQFKPK

SEQ ID NO: 21 - DNA, 23 bp, Artificial Sequence
  Oligonucleotide primer
aaygtnaaygcnathaaraargg

SEQ ID NO: 22 - DNA, 21 bp, Artificial Sequence
  Oligonucleotide primer
ytcrtanacrgcrtgngcntg

SEQ ID NO: 23 - DNA, 23 bp, Artificial Sequence
  Oligonucleotide primer
ggnggncarathathggnaargc

SEQ ID NO: 24 - DNA, 23 bp, Artificial Sequence
  Oligonucleotide primer
tgnsndatdatrtcrtgngcngt

SEQ ID NO: 25 - DNA, 22 bp, Artificial Sequence
  Oligonucleotide primer
gaggaaaggccataggaaaagg

SEQ ID NO: 26 - DNA, 18 bp, Artificial Sequence
  Oligonucleotide primer
actcgccgcactgattac

SEQ ID NO: 27 - DNA, 18 bp, Artificial Sequence
  Oligonucleotide primer
ggggggcagatcattggg

SEQ ID NO: 28 - DNA, 19 bp, Artificial Sequence
  Oligonucleotide primer
ttatgtcatgggccgtact

SEQ ID NO: 29 - DNA, 337 bp, Galleria mellonella
ggtaacatctttattagttatcgtaaaataacagattgtagaaatgaagtttacaggaatattcttcataattatggcgatcattgccctctttatagggtcaaatgaagcggcgcctaaagtcaatgttaatgccattaagaagggaggaaaggccataggaaaaggatttaaagtaatcagtgcggcgagtacagcgcatgacgtctatgaacacattaaaaacagaaggcactaataaaaccaaaaataattatttattttataaggtaattttaagacatataatgtatgttgcaaattattaagtgaaataaaatataaaatattttttgtt

SEQ ID NO: 30 - protein, 32 aa, Galleria mellonella
KVPIGAIKKGGKIIKKGLGVIGAAGTAHEVYS

SEQ ID NO: 31 - DNA, 20 bp, Artificial Sequence
  Oligonucleotide sequence
ccnaargtnccnathggngc

SEQ ID NO: 32 - DNA, 20 bp, Artificial Sequence
  Oligonucleotide Primer
tanacttcrtgngcdgtncc

SEQ ID NO: 33 - DNA, 20 bp, Artificial Sequence
  Oligonucleotide Primer
aggtcttggtgtaattggtg

SEQ ID NO: 34 - DNA, 20 bp, Artificial Sequence
  Oligonucleotide Sequence
gcagcaccaattacaccaag

SEQ ID NO: 35 - DNA, 20 bp, Artificial Sequence
  Oligonucleotide Sequence
taaaaagggtctaggtgtgc

SEQ ID NO: 36 - DNA, 20 bp, Artificial Sequence
  Oligonucleotide Sequence
gcggcgccaagcacacctag

SEQ ID NO: 37 - DNA, 24 bp, Artificial Sequence
  Oligonucleotide Primer
cttcaatcttagtgaaaacttcgc

SEQ ID NO: 38 - DNA, 24 bp, Artificial Sequence
  Oligonucleotide Primer
ggatagtacttcataattatatac

SEQ ID NO: 39 - DNA, 23 bp, Artificial Sequence
  Oligonucleotide Sequence
gttgcaggacttaatacttagtg

SEQ ID NO: 40 - DNA, 25 bp, Artificial Sequence
  Oligonucleotide Sequence
gagtattttactaataagtatgtgg

SEQ ID NO: 41 - DNA, 35 bp, Artificial Sequence
  Oligonucleotide Primer
ctcgagaacaatgaagtttacaggaatattcttca

SEQ ID NO: 42 - DNA, 39 bp, Artificial Sequence
  Oligonucleotide Primer
tctagattagtgccttctgtttttaatgtgttcatagac

SEQ ID NO: 43 - DNA, 19 bp, Artificial Sequence
  Oligonucleotide Primer
cgccagaggacccctaaac

SEQ ID NO: 44 - DNA, 21 bp, Artificial Sequence
  Oligonucleotide Primer
atcgatgccagaaccaagaga

SEQ ID NO: 45 - DNA, 42 bp, Artificial Sequence
  Oligonucleotide Primer
tcgaaggagatgccaccatgaagtttacaggaatattcttca

SEQ ID NO: 46 - DNA, 33 bp, Artificial Sequence
  Oligonucleotide Primer
ttagtgccttctgtttttaatgtgttcatagac

SEQ ID NO: 47 - protein, 63 aa, Galleria mellonella
MKLTGLFFMIMAMLALFVGAGQADPKVPIGAIKKGGKIIKKGLGVIGAAGTAHEVYSHVKNRH

SEQ ID NO: 48 - protein, 38 aa, Galleria mellonella
KVPIGAIKKGGKIIKKGLGVIGAAGTAHEVYSHVKNRH

SEQ ID NO: 49 - DNA, 375 bp, Galleria mellonella
gtaacagtaccaccgtgtacagtcgcagtagttagtcttcaatcttagtgaaaacttcgcttctctttatcaaccatgaagctgaccggtctatttttcatgatcatggcgatgctcgccctgtttgttggcgctggtcaagccgaccctaaggtgcccattggcgccatcaagaagggtggcaaaattattaaaaaaggtcttggtgtaattggtgccgctggtacagcgcatgaagtatatagccacgtcaagaacaggcattagattcttgaagaatatatagtatataattatgaagtactatccttttgtatatgtgactaagtgcataatgtaaagtcaaatgaaatatatattatttatcctcgtgcc

SEQ ID NO: 50 - DNA, 192 bp, Galleria mellonella
atgaagctgaccggtctatttttcatgatcatggcgatgctcgccctgtttgttggcgctggtcaagccgaccctaaggtgcccattggcgccatcaagaagggtggcaaaattattaaaaaaggtcttggtgtaattggtgccgctggtacagcgcatgaagtatatagccacgtcaagaacaggcattag

SEQ ID NO: 51 - DNA, 117 bp, Galleria mellonella
aaggtgcccattggcgccatcaagaagggtggcaaaattattaaaaaaggtcttggtgtaattggtgccgctggtacagcgcatgaagtatatagccacgtcaagaacaggcattag

SEQ ID NO: 52 - protein, 63 aa, Galleria mellonella
MKLTGLFLMIMAVLALFVGAGQADPKVPIGAIKKGGKIIKKGLGVLGAAGTAHEVYNHVRNRQ

SEQ ID NO: 53 - protein, 38 aa, Galleria mellonella
KVPIGAIKKGGKIIKKGLGVLGAAGTAHEVYNHVRNRQ

SEQ ID NO: 54 - DNA, 462 bp, Galleria mellonella
acttcattgtgtacagttgcaggacttaatacttagtgaactacttactcctcgttaccaaccatgaagctgaccggtctatttctcatgatcatggcggtgctcgcgctgtttgttggcgctggtcaagccgaccctaaggtgcccattggcgctatcaagaagggcggcaaaattattaaaaagggtctaggtgtgcttggcgccgcgggcacagcgcacgaagtgtacaaccacgttaggaacaggcagtaacgtcatgcgtgattgttgtacatacagtacttacaatacgatttgtcttggctgtgatatatctttagataaattaatttataataccacatacttattagtaaaatactcaaatatattgattatagatacattaataaatattaattattacaatattttgtttttatgtacaatgcgaatagattctaccctctgcctcgtgcc

SEQ ID NO: 55 - DNA, 192 bp, Galleria mellonella
atgaagctgaccggtctatttctcatgatcatggcggtgctcgcgctgtttgttggcgctggtcaagccgaccctaaggtgcccattggcgctatcaagaagggcggcaaaattattaaaaagggtctaggtgtgcttggcgccgcgggcacagcgcacgaagtgtacaaccacgttaggaacaggcagtaa

SEQ ID NO: 56 - DNA, 117 bp, Galleria mellonella
aaggtgcccattggcgctatcaagaagggcggcaaaattattaaaaagggtctaggtgtgcttggcgccgcgggcacagcgcacgaagtgtacaaccacgttaggaacaggcagtaa

SEQ ID NO: 57 - protein, 67 aa, Spodoptera exigua
MKLTKVFVIVIVVVALLVPSEAAPGKIPVKAIKKAGTAIGKGLRAINIASTAHDVYSFFKPKHKKKH

SEQ ID NO: 58 - protein, 54 aa, Hyblaea puera
AMSLVSCSTAAPAKIPIKAIKTVGKAVGKGLRAINIASTANDVFNFLKPKKRKH

SEQ ID NO: 59 - protein, 41 aa, Caligo illioneus
GKIPINAIRKGAKAVGHGLRALNIASTAHDIASAFHRKRKH

SEQ ID NO: 60 - protein, 37 aa, Caligo illioneus
RKIPVEAIKKGASRAWRALDLASTAYDIASIFNRKRE

SEQ ID NO: 61 - protein, 40 aa, Caligo illioneus
GKIPVEALKKGAKVAGRAWRALDLASTAYDIAHLFDRKRN

SEQ ID NO: 62 - protein, 43 aa, Artificial Sequence
  Consensus sequence for Galleria peptides
XKXXXXAIKKGGXXIXKXXXXXXXAXTAHXXXXXXXXXXXXXX